(19) United States
(12) Patent Application Publication
(10) Pub. No.: US 2009/0118825 A1
(43) Pub. Date: May 7, 2009

(54) METHOD AND APPARATUS FOR IMPROVING MITRAL VALVE FUNCTION

(76) Inventors: Jonathan Rourke, Belmont, MA (US); Michael Atlas, Arlington, MA (US); Steve Blacker, Framingham, MA (US); Terry Barnes, Somerville, MA (US); Francis Gurrie, Haverhill, MA (US); Christopher Hutchins, Londonderry, NH (US)

Correspondence Address: Mark J. Pandiscio;Pandiscio & Pandiscio, P.C., 470 Totten Pond Road, Waltham, MA, 02451, US

(21) Appl. No.: 12/260,858

(22) Filed: Oct. 29, 2008

Related U.S. Application Data
(63) Continuation-in-part of application No. 11/582,157, filed on Oct. 17, 2006.
Application No. 11/582,157 is a continuation-in-part of application No. 11/708,662, filed on Feb. 20, 2007.
Application No. 11/708,662 is a continuation-in-part of application No. 11/286,906, filed on Nov. 23, 2005.

(51) Int. Cl. A61F 2/24

(52) U.S. Cl. 623/2.11; 623/2.36

(57) ABSTRACT

A method and apparatus for reducing mitral regurgitation. The apparatus is inserted into the coronary sinus of a patient in the vicinity of the posterior leaflet of the mitral valve, the apparatus being adapted to straighten the natural curvature of at least a portion of the coronary sinus in the vicinity of the posterior leaflet of the mitral valve, whereby to move the posterior annulus anteriorly and thereby improve leaflet coaptation and reduce mitral regurgitation.

### REFERENCE TO PENDING PRIOR PATENT APPLICATIONS

This patent application:

(i) is a continuation-in-part of pending prior U.S. patent application Ser. No. 11/582,157, filed Oct. 17, 2006 by Jonathan Rourke et al. for METHOD AND APPARATUS FOR IMPROVING MITRAL VALVE FUNCTION (Attorney's Docket No. VIA-43 CON);

(ii) is a continuation-in-part of pending prior U.S. patent application Ser. No. 11/708,662, filed Feb. 20, 2007 by Jonathan M. Rourke et al. for METHOD AND APPARATUS FOR IMPROVING MITRAL VALVE FUNCTION (Attorney's Docket No. VIA-48 CON); and

(iii) is a continuation-in-part of pending prior U.S. patent application Ser. No. 11/286,906, filed Nov. 23, 2005 by Jonathan M. Rourke et al. for METHOD AND APPARATUS FOR IMPROVING MITRAL VALVE FUNCTION (Attorney's Docket No. VIA-49).

The three above-identified patent applications are hereby incorporated herein by reference.

### FIELD OF THE INVENTION

This invention relates to surgical methods and apparatus in general, and more particularly to surgical methods and apparatus for improving mitral valve function.

### BACKGROUND OF THE INVENTION

The mitral valve is located in the heart between the left atrium and the left ventricle. A properly functioning mitral valve permits blood to flow from the left atrium to the left ventricle when the left ventricle expands (i.e., during diastole), and prevents the regurgitation of blood from the left ventricle back into the left atrium when the left ventricle contracts (i.e., during systole).

In some circumstances the mitral valve may fail to function properly, such that regurgitation may occur. By way of example, mitral regurgitation is a common occurrence in patients with heart failure. Mitral regurgitation in patients with heart failure is typically caused by changes in the geometric configurations of the left ventricle, papillary muscles and mitral annulus. These anatomical changes frequently result in incomplete coaptation of the mitral leaflets during systole, resulting in mitral regurgitation.

Mitral regurgitation is generally treated by plicating the mitral valve annulus so as to correct the shape of the distended annulus and restore the original geometry of the mitral valve annulus.

More particularly, current surgical practice for mitral valve repair generally requires that the distended mitral valve annulus be restored by surgically opening the left atrium and then fixing sutures, or more commonly sutures in combination with a support ring, to the internal surface of the annulus; this structure is then used to draw the annulus, in a pursestring-like fashion, back into its proper configuration, thereby improving leaflet coaptation and reducing mitral regurgitation.

This method of mitral valve repair, generally referred to as “annuloplasty”, effectively reduces mitral regurgitation in heart failure patients. This, in turn, reduces the symptoms associated with heart failure, improves the patient's quality of life and increases patient longevity. Unfortunately, however, such mitral valve surgery is highly traumatic for the patient, i.e., it generally involves the use of general anesthesia, the creation of a chest wall incision, the application of cardiopulmonary bypass, the initiation of cardiac and pulmonary arrest, the creation of an incision into the heart itself so as to gain access to the mitral valve, etc. Due to the traumatic nature of such conventional mitral valve surgery, and the risks associated therewith, most heart failure patients are poor candidates for such surgery. Thus, a less invasive means to reconfigure a distended mitral valve annulus in heart failure patients, and thereby increase leaflet coaptation and reduce mitral regurgitation, would make therapy available to a much larger population of patients.

Mitral regurgitation also occurs in approximately 20% of patients who experience acute myocardial infarction. In addition, mitral regurgitation is the primary cause of cardiogenic shock in approximately 10% of patients who develop severe hemodynamic instability in the setting of acute myocardial infarction. Patients with mitral regurgitation and cardiogenic shock generally have a high mortality rate, i.e., approximately 50%. Elimination of mitral regurgitation in these patients would, therefore, also provide significant benefit for the patient. Unfortunately, however, patients with acute mitral regurgitation complicating acute myocardial infarction are particularly high-risk surgical candidates, and are therefore poor candidates for a traditional annuloplasty procedure. Thus, a minimally invasive means to effect a temporary reduction or elimination of mitral regurgitation in these critically ill patients would afford them the time to recover from the myocardial infarction or other acute life-threatening events, and make them better candidates for other medical, interventional or surgical therapy.

### SUMMARY OF THE INVENTION

These and other objects are addressed by the provision and use of the present invention, which comprises a novel method and apparatus for reducing mitral regurgitation.

In one form of the invention, there is provided treatment apparatus for reducing mitral regurgitation, the treatment apparatus comprising a treatment section which is sized and shaped for disposition within the coronary sinus of a patient, with the treatment section being formed so as to: (i) be somewhat more rigid than the anatomical tissue surrounding the posterior leaflet of the mitral valve; and (ii) have a shape somewhat straighter than the natural curvature of the coronary sinus in the vicinity of the posterior leaflet of the mitral valve; and (iii) have a length sufficient to span from the trigone CSO strongpoint to the trigone AIV strongpoint; such that when the treatment apparatus is placed in the coronary sinus of the patient, the treatment section applies posteriorly-directed forces to the outer wall of the coronary sinus in the vicinity of the trigone CSO strongpoint and the trigone AIV strongpoint, and the treatment section applies an anteriorly-directed force to the posterior annulus of the mitral valve, whereby to reconfigured the mitral valve and thereby reduce mitral regurgitation.

And in another form of the invention, there is provided a method for reducing mitral regurgitation, the method comprising the steps of:

providing treatment apparatus comprising a treatment section which is sized and shaped for disposition within the coronary sinus of a patient, with the treatment section being formed so as to: (i) be somewhat more rigid than the anatomical tissue surrounding the posterior leaflet of the mitral valve; and (ii) have a shape somewhat straighter than the natural curvature of the coronary sinus in the vicinity of the posterior leaflet of the mitral valve; and (iii) have a length sufficient to span from the trigone CSO strongpoint to the trigone AIV strongpoint; and

deploying the treatment apparatus in the coronary sinus of the patient, such that the treatment section applies posteriorly-directed forces to the outer wall of the coronary sinus in the vicinity of the trigone CSO strongpoint and the trigone AIV strongpoint, and the treatment section applies an anteriorly-directed force to the posterior annulus of the mitral valve, whereby to reconfigure the mitral valve and thereby reduce mitral regurgitation.

And in another form of the invention, there is provided an assembly for reducing mitral regurgitation, the assembly comprising:

a delivery catheter comprising:
a shaft formed out of a material sufficiently flexible to assume a first configuration generally conforming to a coronary sinus upon insertion of the shaft into the coronary sinus, and to assume a straighter second configuration when biased toward the straighter configuration, the shaft having at least one lumen extending lengthwise therethrough;
a handle slidably mounted on the shaft, the handle comprising an end plate having at least one opening therein for alignment with the at least one lumen of the shaft; and
a collet mechanism attached to the handle for fixing the position of the handle relative to the shaft;

at least one treatment rod comprising a treatment section, a rod shaft and a peripheral enlargement disposed at the proximal end of the rod shaft;
the treatment section and the rod shaft being sized to fit within a lumen in the shaft and an opening in the end plate of the handle, and the peripheral enlargement being sized larger than the opening in the end plate of the handle so as to limit distal movement of the treatment rod relative to the delivery catheter;
the treatment section being formed so as to (i) be more rigid than the anatomical tissue surrounding the posterior leaflet of the mitral valve; and (ii) have a shape straighter than the shape of the coronary sinus in the vicinity of the posterior leaflet of the mitral valve; and (iii) have an adequate length relative to the radius of curvature of the coronary sinus, such that when the at least one treatment rod is advanced down the lumen while the shaft is positioned in the coronary sinus adjacent to the posterior leaflet of the mitral valve, the treatment section will impart a straightening force to the wall of the coronary sinus, whereby to move the posterior annulus anteriorly so as to improve leaflet coaptation and, as a result, reduce mitral regurgitation; and

a pullback device mountable to the delivery catheter for adjusting the position of the handle along the shaft before the collet mechanism is used to fix the position of the handle along the shaft.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts and further wherein:

FIG. 1 is a schematic view of portions of the human vascular system;

FIG. 2 is a schematic view of portions of the human heart;

FIG. 3 is a schematic view showing a novel annuloplasty system for use in improving mitral valve function;

FIG. 4 is a schematic view like that of FIG. 3, but with some of the components broken along their length so as to improve visualization of various aspects of the components;

FIGS. 5 and 6 are schematic views showing the delivery catheter of the annuloplasty system, with FIG. 5 being a side view of the delivery catheter and with FIG. 6 being a distal end view of the delivery catheter, and with the delivery catheter having alignment stylets disposed in its working lumens, as will hereinafter be discussed in further detail;

FIG. 7 is a cross-sectional view of the shaft of the delivery catheter;

FIGS. 8 and 9 are schematic views showing the distal end of the shaft of the delivery catheter;

FIGS. 10-15 are schematic views showing the traction mechanism of the delivery catheter;

FIGS. 16-20 are schematic views showing the handle of the delivery catheter;

FIG. 21 is a schematic view showing the distal end of a diagnostic rod and/or treatment rod of the annuloplasty system;

FIG. 22 is a schematic view showing the proximal end of a diagnostic rod;

FIG. 23 is a schematic view showing the proximal end of a treatment rod;

FIGS. 24-28 are schematic views showing preferred constructions for the distal end of a diagnostic rod and/or treatment rod of the annuloplasty system;

FIGS. 29-47 are schematic views showing the pullback device of the annuloplasty system and how it may be used to properly position a treatment rod within the delivery catheter; and

FIGS. 48-53 are schematic views showing the annuloplasty system of the present invention disposed in the coronary sinus of a patient so as to improve mitral valve function.

### DETAILED DESCRIPTION OF THE PREFERRED EMBODIMENTS

### Overview

The coronary sinus is the largest vein in the human heart. During a large portion of its course in the atrioventricular groove, the coronary sinus extends adjacent to the left atrium of the heart, e.g., for a distance of approximately 5-10 cm. Significantly, for a portion of its length, e.g., approximately 7-9 cm, the coronary sinus extends substantially adjacent to the posterior perimeter of the mitral annulus.

The present invention takes advantage of this anatomical characteristic in order to treat mitral regurgitation in a distended mitral valve. More particularly, by deploying novel apparatus in the coronary sinus of a patient, adjacent to the posterior leaflet of the mitral valve, the distended curvature of the coronary sinus can be corrected in the vicinity of the posterior leaflet of the mitral valve, whereby to move the posterior annulus anteriorly so as to improve leaflet coaptation and, as a result, reduce mitral regurgitation.

### Patient Anatomy

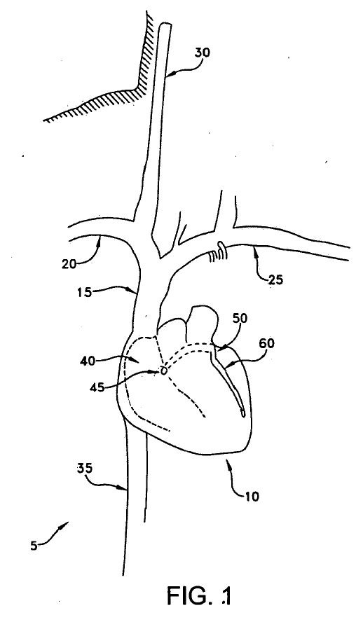

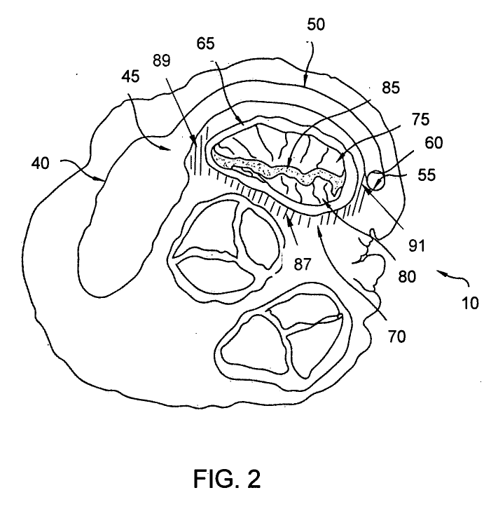

Looking now at FIGS. 1 and 2, there are shown aspects of the cardiovascular system 5 of a patient. More particularly, cardiovascular system 5 generally comprises the heart 10, the superior vena cava 15, the right subclavian vein 20, the left subclavian vein 25, the jugular vein 30 and the inferior vena cava 35. Superior vena cava 15 and inferior vena cava 35 communicate with the heart's right atrium 40. The coronary ostium 45 (also sometimes referred to herein as the CSO) leads to the coronary sinus 50. At the far end 55 (FIG. 2) of coronary sinus 50, the vascular structure leads to the vertically-descending anterior interventricular vein (“AIV”) 60 (FIGS. 1 and 2). For the purposes of the present invention, it can generally be convenient to consider the term “coronary sinus” to mean the vascular structure extending between coronary ostium 45 and AIV 60.

As seen in FIG. 2, between coronary ostium 45 and AIV 60, coronary sinus 50 generally extends substantially adjacent to the posterior perimeter of the annulus 65 of the mitral valve 70. Mitral valve 70 comprises a posterior leaflet 75 and an anterior leaflet 80. In the case of a regurgitant mitral valve, posterior leaflet 75 and anterior leaflet 80 will generally fail to properly coapt at systole, thereby leaving an intervening gap 85 which can permit the undesired regurgitation of blood to occur.

It should also be appreciated that, within the heart itself, there exists an internal fibrous “skeleton” which provides a supporting structure for the heart valves and other coronary tissue. In the region of the mitral valve, and still looking now at FIG. 2, the fibrous structure 87 includes a “CSO strongpoint” trigone 89 and an “AIV strongpoint” trigone 91, in the sense that the coronary sinus is strongly connected to the fibrous structure of the heart at these two locations.

### Annuloplasty System 100

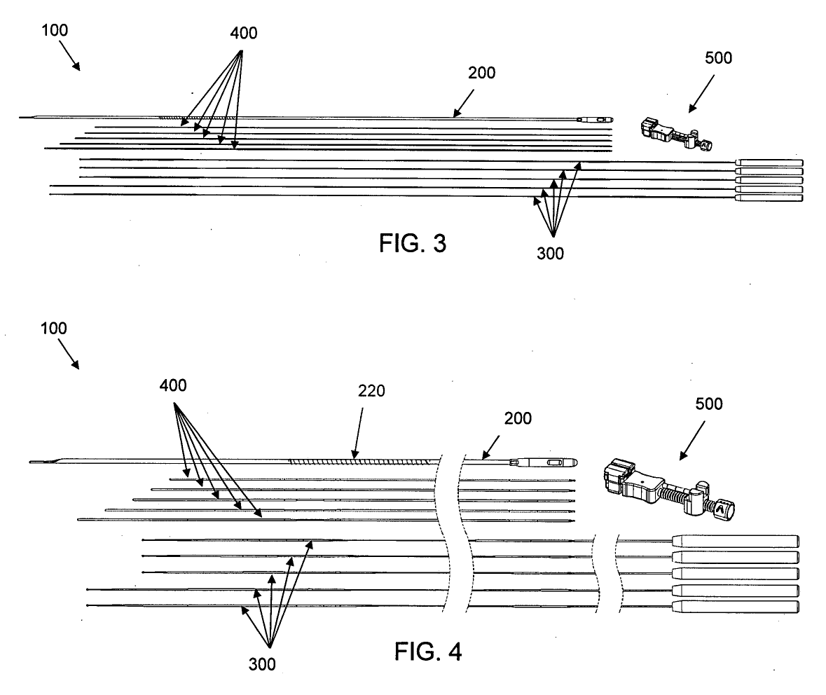

Looking next at FIGS. 3 and 4, there is shown an annuloplasty system 100 which comprises one preferred form of the present invention. Annuloplasty system 100 generally comprises a delivery catheter 200 for deployment in the vascular system of a patient, at least one diagnostic rod 300 for temporary positioning within delivery catheter 200 for assessing the appropriate treatment for the patient, at least one treatment rod 400 for permanent positioning within delivery catheter 200 for providing treatment for the patient, and a pullback device 500 for proper positioning of treatment rod 400 within delivery catheter 200, all as will hereinafter be discussed in further detail.

### Delivery Catheter 200

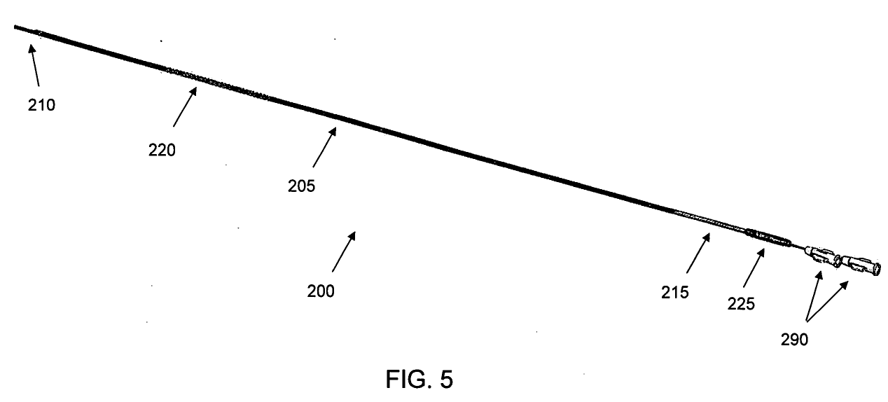

Looking next at FIG. 5, in one preferred form of the present invention, delivery catheter 200 generally comprises a shaft 205 having a distal end 210 and a proximal end 215. A traction mechanism 220 is secured to the exterior of shaft 205 near distal end 210. A handle 225 is attached to proximal end 215 of shaft 205 as will hereinafter be discussed.

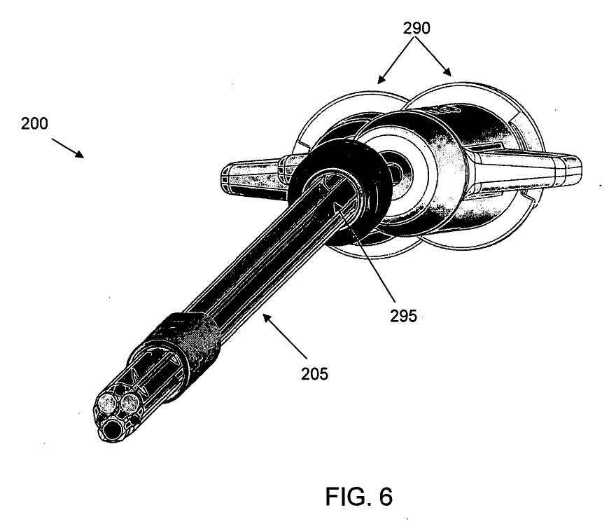

Looking next at FIGS. 6-9, shaft 205 is preferably formed out of a relatively flexible material, such that delivery catheter 200 can traverse the vascular system of a patient so as to extend from an incision site (e.g., in the upper torso), down the superior vena cava, through the right atrium of the heart, through the coronary sinus, and then into the AIV, so as to effectively form a delivery “corridor” into and through the coronary sinus, as will hereinafter be discussed in further detail. In addition, shaft 205 is preferably formed out of a relatively low friction material, such that delivery catheter 200 can be advanced easily over a guidewire, and such that diagnostic rods 300, treatment rods 400, and/or other rods, wires and the like can be easily advanced into, and easily withdrawn from, delivery catheter 200, as will hereinafter be discussed in further detail. In one preferred form of the present invention, shaft 205 is formed out of Teflon.

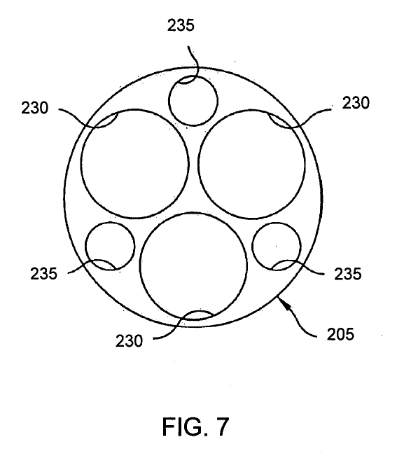

As seen in FIGS. 6-9, shaft 205 comprises at least one, and preferably a plurality, of working lumens 230 (FIG. 7) extending from its proximal end 215 to its distal end 210. Working lumens 230 may all have the same diameter as one another, or they may have different diameters from one another. In one preferred construction, three identical working lumens 230, equally disposed about the center axis of shaft 205, extend substantially all the way from proximal end 215 of shaft 205 to distal end 210 of shaft 205.

Working lumens 230 are intended to selectively receive diagnostic rods 300 for assessing the appropriate treatment for the patient, and treatment rods 400 for providing treatment for the patient, as will hereinafter be discussed in further detail.

Still looking now at FIGS. 6 and 7, shaft 205 may also comprise at least one, and preferably a plurality, of auxiliary lumens 235 (FIG. 7) extending from its proximal end 215 to its distal end 210. Auxiliary lumens 235 may all have the same diameter as one another, or they may have different diameters from one another. Furthermore, one or more of auxiliary lumens 235 may have the same diameter as one or more of working lumens 230. In one preferred construction, three identical auxiliary lumens 235, equally disposed about the center axis of shaft 205 and having a diameter less than the diameter of working lumens 230, extend substantially all the way from proximal end 215 of shaft 205 to distal end 210 of shaft 205.

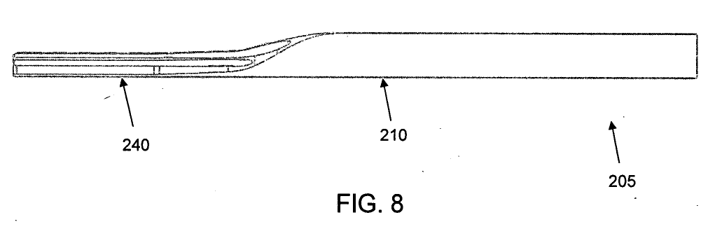

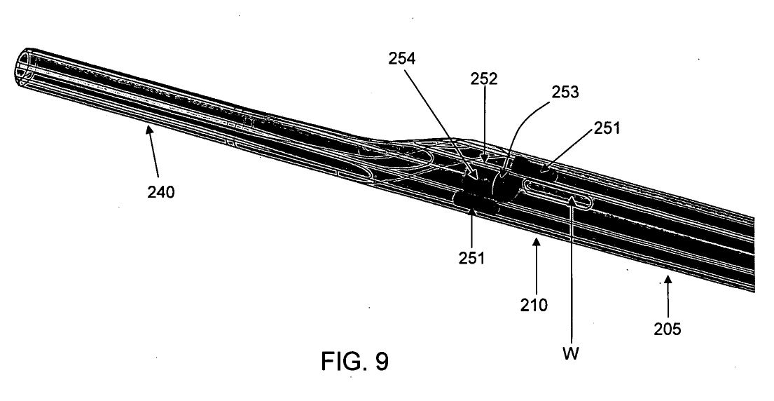

As seen in FIGS. 8 and 9, distal end 210 of shaft 205 is preferably cut away as shown so as to create a distal finger 240 having a reduced diameter vis-à-vis the remainder of shaft 205. Distal finger 240 is preferably sized so as to include at least one complete working lumen 230. Due to its reduced diameter, distal finger 240 is relatively pliable so as to provide an atraumatic front tip for the distal end of shaft 205. In addition, the reduced diameter of distal finger 240 facilitates advancement of delivery catheter 200 through the torturous vascular system of the patient, since it can effectively function as a tapered end for shaft 205.

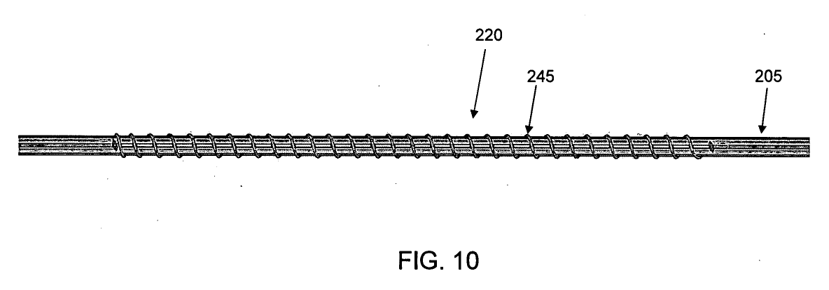

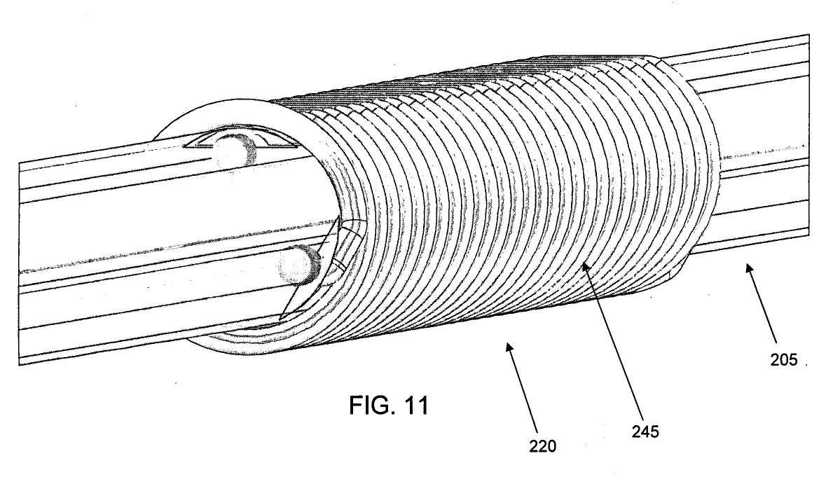

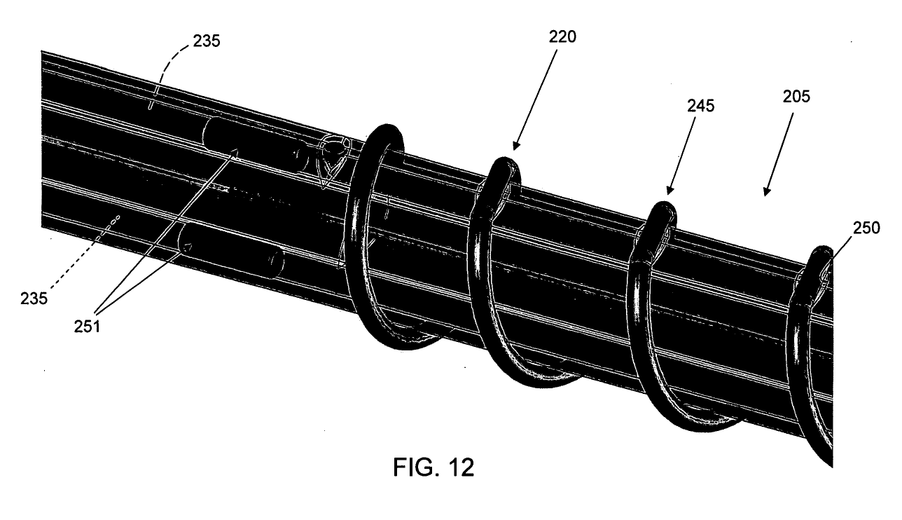

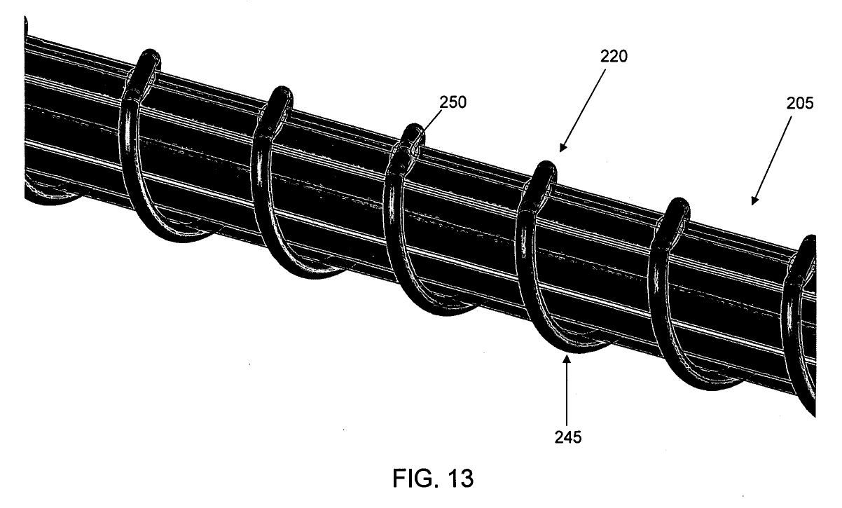

Looking next at FIGS. 4, 5 and 10-15, traction mechanism 220 preferably comprises a helical coil 245 disposed on the outer surface of shaft 205. In one preferred form of the invention, helical coil 245 comprises a suture-like filament which is wrapped about the exterior of shaft 205, seating in notches 250 (FIGS. 12 and 13) formed in the outer surface of shaft 205. The free ends of helical coil 245 are preferably disposed in at least one auxiliary lumen 235. This construction facilitates securing helical coil 245 to shaft 205. More particularly, it will be appreciated that shaft 205 is preferably formed out of a relatively low friction material (e.g., Teflon) which can be difficult to adhere to. By securing helical coil 245 to shaft 205 by using at least one auxiliary lumen 235, effective anchoring of helical coil 245 can be achieved without requiring helical coil 245 to physically adhere to the surface of shaft 205.

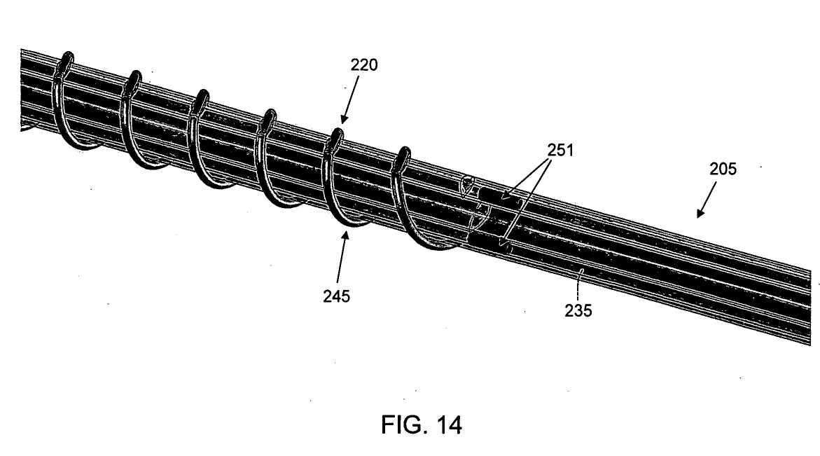

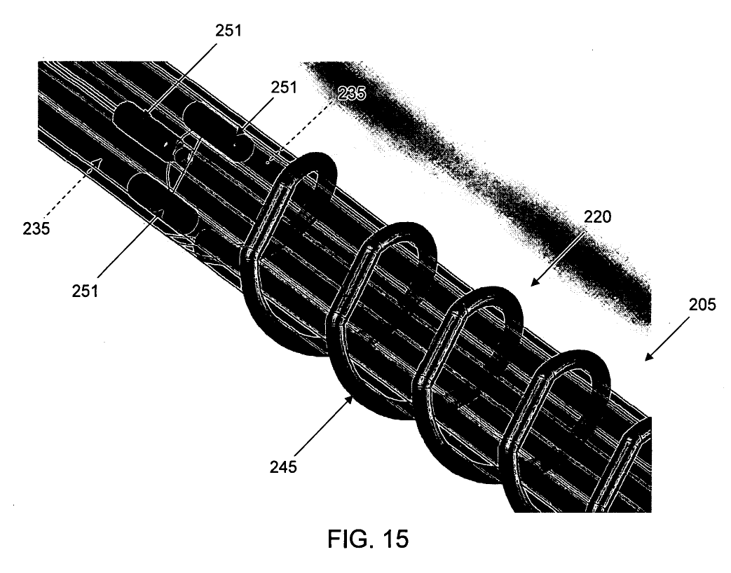

In order to facilitate visualization of delivery catheter 200 after it has been placed within the vascular system of the patient, it is preferred that radio-opaque markers 251 be disposed within one or more of auxiliary lumens 235 (see FIGS. 9, 12, 14 and 15). Preferably at least two sets of radio-opaque markers 251 are provided, one set adjacent to the distal end of traction mechanism 220 (FIG. 12) and one set adjacent to the proximal end of traction mechanism 220 (FIGS. 14 and 15).

Preferably two of the working lumens 230 are sealed with a plug 252 (FIG. 9) adjacent to the distal end of shaft 205. Plugs 252 are positioned sufficiently far down working lumens 230 (i.e., sufficiently distal within working lumens 230) that they will not interfere with a diagnostic rod 300 or a treatment rod 400 which may be positioned within a working lumen, as will hereinafter be discussed. In one preferred form of the present invention, plug 252 comprises a radio-opaque marker 253 and a urethane body 254. By etching the distal end of the two working lumens which are to receive plugs 252, the urethane body 254 can adhere to shaft 205. And in one preferred form of the present invention, a window W (FIG. 9) is provided between the two working lumens which are to receive plugs 252, with window W being positioned proximal to plugs 252. Window W permits fluid to communicate between the two working lumens. This can be important where the two working lumens are to be flushed, and can minimize the formation of a pressure head in front of an element (e.g., a diagnostic rod 300 or a treatment rod 400) advancing down a working lumen.

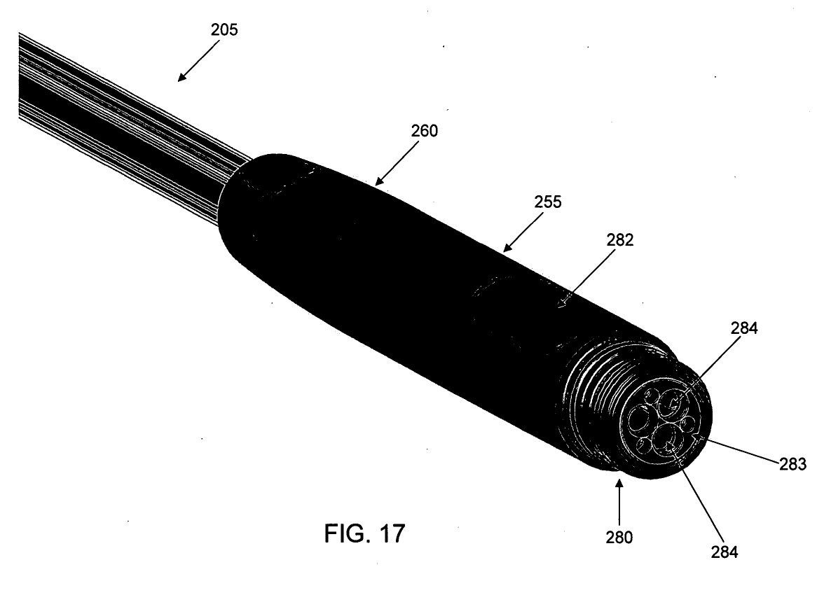

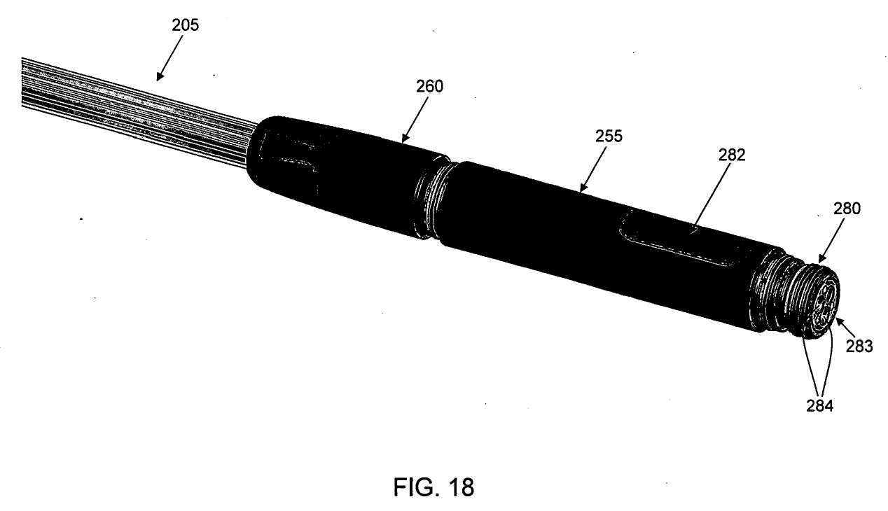

Looking next at FIGS. 5 and 16-20, handle 225 comprises a body 255, a distal cap 260 and a proximal cap 265.

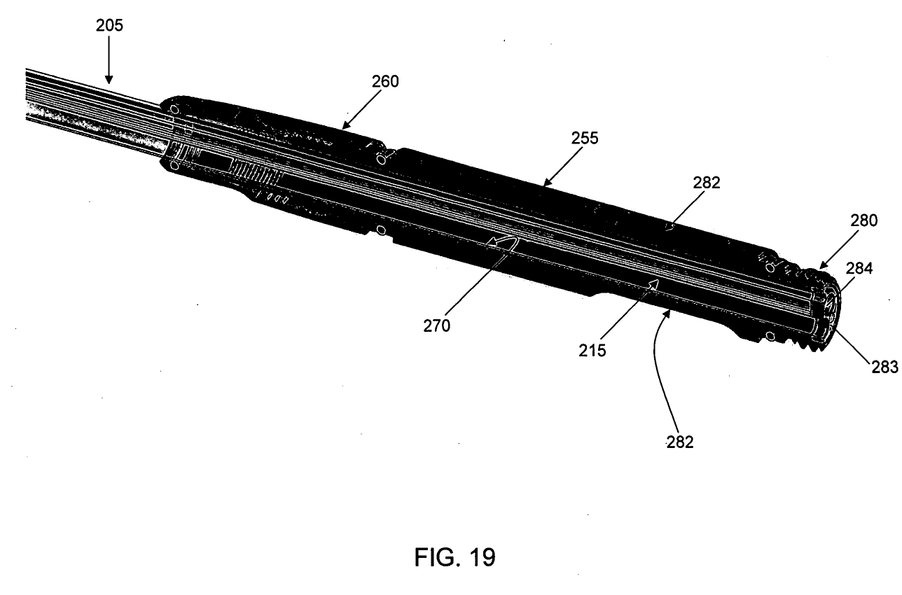

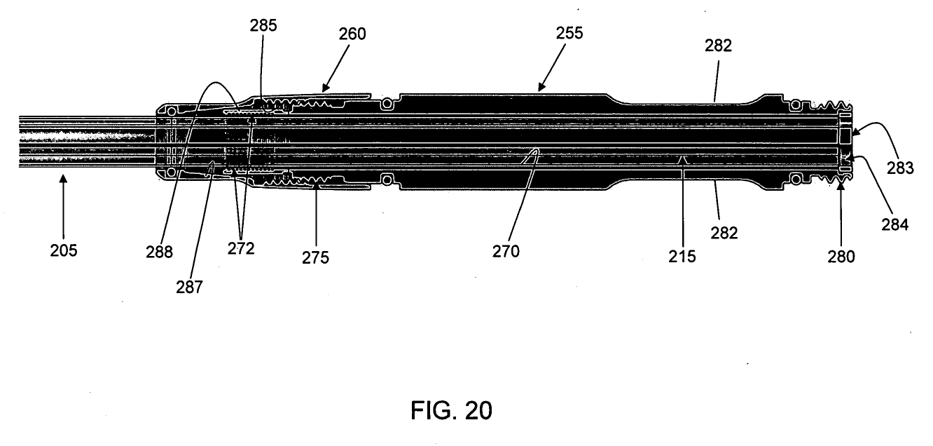

Handle body 255 has a central lumen 270 (FIG. 19) extending therethrough for receiving proximal end 215 of shaft 205. Central lumen 270 is sized so as to be slightly larger than shaft 205, so that handle body 255 can slide on shaft 205 unless it is otherwise locked to the shaft. Handle body 255 is segmented into at least two, and preferably four, fingers 272 (FIG. 20) at its distal end so as to be selectively compressible onto the outer surface of shaft 205. Distal screw threads 275 are formed on handle body 255 adjacent to where handle body 255 is segmented into fingers 272. Proximal screw threads 280 are formed on handle body 255 adjacent to the proximal end of the handle body. A pair of diametrically-opposed flats 282 is formed on the outer surface of handle body 255. A plate 283, having openings 284 formed therein, is disposed at the proximal end of handle body 255. Openings 284 correspond to working lumens 230 in number, size and relative disposition.

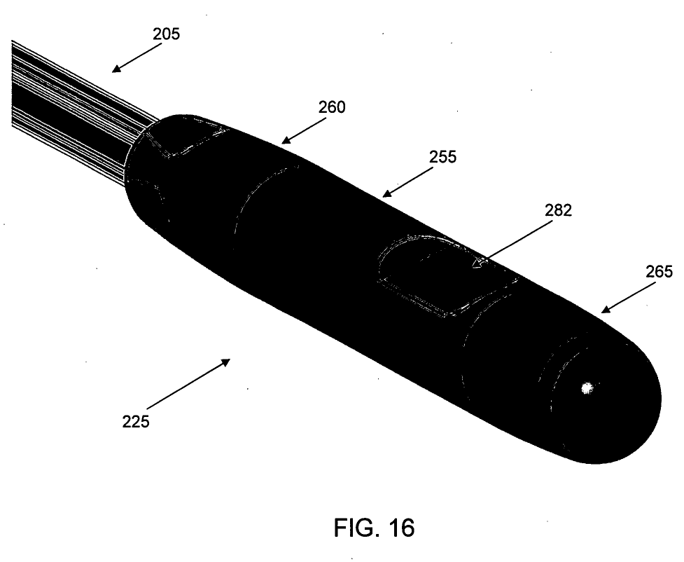

Distal cap 260 (FIG. 16) is sized to selectively fit over the distal end of handle body 255 and includes screw threads 285 (FIG. 20) which mate with distal screw threads 275 of handle body 255. The inner surface 287 of distal cap 260 mates with the segmented outer surfaces 288 of fingers 272 so that when distal cap 260 is screwed onto handle body 255, fingers 272 of handle body 255 are squeezed down onto the outer surface of shaft 205, whereby to grip shaft 205 and thereby secure shaft 205 to handle 225. Thus it will be seen that when distal cap 260 is not secured to handle body 255, body 255 and shaft 205 are free to move relative to one another. However, when distal cap 260 is secured to handle body 255, fingers 272 will lock body 255 to shaft 205.

Proximal cap 265 (FIG. 16) is sized to fit over the proximal end of handle body 255 and includes internal screw threads (not shown) which mate with proximal screw threads 280 (FIG. 20) of handle body 255.

Inasmuch as handle body 255 is free to move relative to shaft 205 when distal cap 260 is not secured to handle body 255, it is generally preferred that delivery catheter 200 includes a pair of alignment stylets 290 (FIG. 6) so as to ensure that working lumens of shaft 205 remain aligned with openings 284 (FIG. 19) in plate 283 while alignment stylets 290 are disposed in working lumens 230. Preferably the shafts 295 (FIG. 6) of alignment stylets 290 are cannulated so as to permit flushing of the working lumens while alignment stylets 290 are in place, as will hereinafter be discussed in further detail.

### Diagnostic Rods 300 and Treatment Rods 400

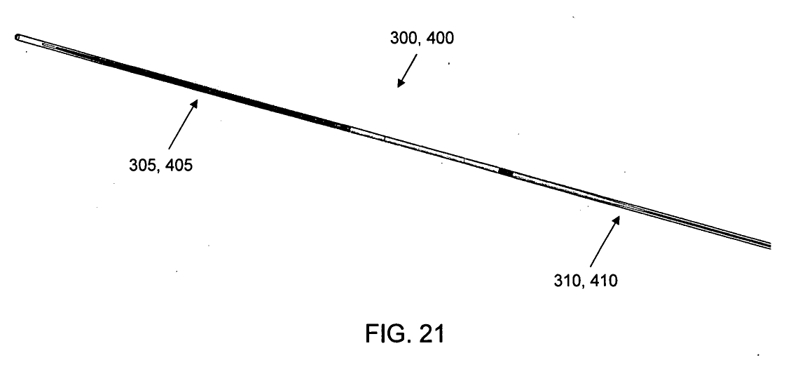

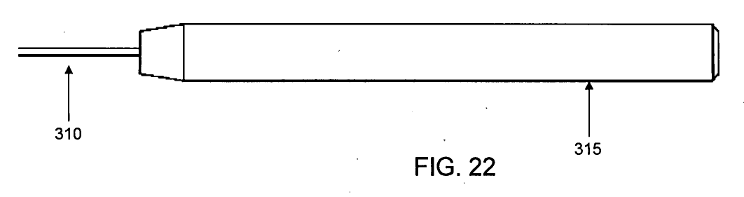

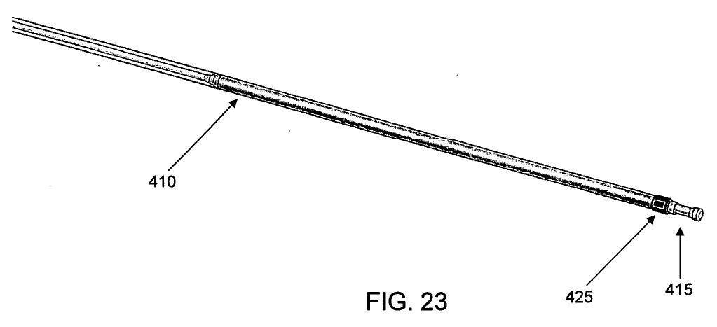

Looking next at FIGS. 3, 4 and 21-28, each of diagnostic rods 300 and treatment rods 400 comprises a treatment section 305, 405, respectively, formed at the distal end of a shaft 310, 410, respectively. The treatment sections 305 of diagnostic rods 300 are preferably identical to the treatment sections 405 of treatment rods 400. Shafts 310 of diagnostic rods 300 are preferably identical to shafts 410 of treatment rods 400, except that shaft 310 is preferably longer than shaft 410, and shaft 310 terminates in a handle 315 (FIG. 22) whereas shaft 410 terminates in a proximal tip 415 (FIG. 23). In one preferred form of the invention, and as shown in FIGS. 3 and 4, a kit comprising a plurality of diagnostic rods 300 and a plurality of treatment rods 400 is provided, with diagnostic rods 300 coming in a range of different shaft lengths and with treatment rods 400 coming in a range of different shaft lengths, and with each diagnostic rod 300 having a treatment rod 400 with a corresponding length.

Treatment sections 305, 405 are formed so as to be (i) somewhat more rigid than the anatomical tissue surrounding the posterior leaflet of the mitral valve; and (ii) have a shape somewhat straighter than the shape of the coronary sinus in the vicinity of the posterior leaflet of the mitral valve; and (iii) have a length at least as long as the distance between the coronary ostium and the AIV; such that when a diagnostic rod 300 or a treatment rod 400 is positioned in a working lumen 230 of shaft 205 after delivery catheter 200 has been positioned in the coronary sinus of a patient adjacent to the posterior leaflet of the mitral valve, treatment sections 305, 405 will impart a straightening force to the wall of the coronary sinus, whereby to move the posterior annulus anteriorly so as to improve leaflet coaptation and, as a result, reduce mitral regurgitation, as will hereinafter be discussed in further detail. In particular, when a diagnostic rod 300 or a treatment rod 400 is positioned in the coronary sinus of the patient, the distal and proximal ends of the treatment section of the rod will apply posteriorly-directed forces to the side wall of the coronary sinus and the intermediate portion of the treatment section of the rod will apply an anteriorly-directed force to the annulus of the mitral valve, whereby to effect the desired valve remodeling.

In other words, treatment sections 305, 405 of diagnostic rods 300 and treatment rods 400 have a size and degree of straightness such that, when placed into the curved coronary sinus, the treatment sections 305, 405 cannot be accommodated by the coronary sinus without causing a change in the geometry of either the coronary sinus, or treatment sections 305, 405, or both—and, by making treatment sections 305, 405 somewhat more rigid than the opposing tissue, such deployment of treatment sections 305, 405 in the coronary sinus will cause a change in the geometry of the tissue, so as to adjust the shape of the mitral valve, whereby to reduce mitral regurgitation, as will hereinafter be discussed in further detail.

Significantly, it has been found that it is important that treatment sections 305, 405 be at least as long as the distance between the coronary ostium and the AIV, so that the distal end of the treatment section can apply its posteriorly-directed force to the coronary sinus in the area of the relatively tough fibrous tissue adjacent to the AIV (i.e., in the area of the aforementioned trigone AIV strongpoint 91, shown in FIG. 2), and so that the proximal end of the treatment section can apply its posteriorly-directed force to the coronary sinus in the area of the relatively tough fibrous tissue adjacent to the coronary ostium (i.e., in the area of the aforementioned trigone CSO strongpoint 89, shown in FIG. 2), whereby to enable the intermediate portion of the treatment rod to apply a sufficient anteriorly-directed force on the annulus of the mitral valve, as will hereinafter be discussed in further detail. This approach provides strong tissue structures for accommodating the substantial forces applied by the distal and proximal ends of treatment sections 305, 405, whereby to permit the desired anteriorly-directed force to be generated and the desired therapeutic effects to be realized, as will hereinafter be discussed in further detail.

In one preferred form of the invention, each of treatment sections 305, 405 comprise a substantially straight rod (in an unstressed condition) which is somewhat flexible, such that the rod will elastically apply a straightening force to the wall of the coronary sinus.

Each treatment section 305, 405 may deliver exactly the same straightening force to the wall of the coronary sinus as every other treatment section 305, 405. Alternatively, treatment sections 305, 405 may be engineered so as to provide differing degrees of straightening force. Thus, in one form of the invention, a kit comprising a variety of different diagnostic rods 300 and treatment rods 400, having different treatment sections 305, 405 providing different degrees of straightening force, may be provided for appropriate selection by the doctor. Differences in straightening force may be achieved through differences in the stiffness of treatment sections 305, 405 (achievable through differences in rod composition, rod diameter, etc.); differences in the length of treatment sections 305, 405; differences in the relative positions of treatment sections 305, 405 relative to one another when using multiple rods (i.e., offsets in relative longitudinal position when using multiple rods); etc.

In one preferred form of the invention, each of treatment sections 305, 405 applies a force to the mitral annulus which is, by itself, adequate to move the mitral annulus only a fraction of the total distance ultimately desired to reduce mitral regurgitation. In this form of the invention, additional diagnostic rods 300 and treatment rods 400 may be deployed in working lumens 230 of delivery catheter 200 so as to supply additional straightening force to the mitral annulus.

Additionally, or as an alternative to the foregoing, the apparatus may be constructed so as to apply an elastic straightening force to the mitral annulus, such that a force which initially moves the mitral annulus only a fraction of the total distance ultimately desired to reduce mitral regurgitation, may dynamically work its therapeutic effect over time as the coronary tissue remodels.

In one preferred form of the invention, each of treatment sections 305, 405 comprises a multizone bar having regions of differing flexibility along the length of the treatment section. As a result, different portions of the mitral annulus may be reconfigured with differing amounts of force so as to achieve improved leaflet coaptation.

By way of example but not limitation, treatment sections 305, 405 can have a constant flexibility along their length; or treatment sections 305, 405 can have a central region of substantially constant flexibility terminating in end regions of higher flexibility; or treatment sections 305, 405 can have a continuously variable flexibility (or flexibilities) along their length, etc.

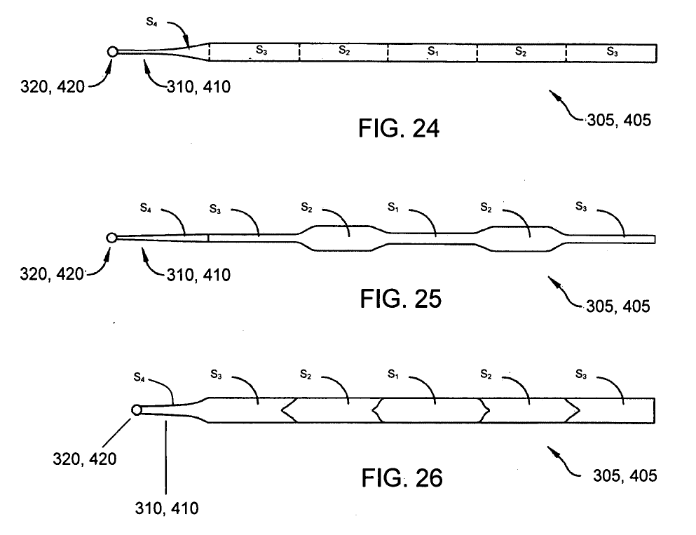

In one particularly preferred form of the invention, each of treatment sections 305, 405 comprises a “5-zone bar” similar to the 5-zone bar disclosed in the aforementioned U.S. patent application Ser. Nos. 11/582,157; 11/708,662; and 11/286,906, e.g., and looking now at FIG. 24, each of treatment sections 305, 405 comprises a central region (or hinge) S₁ having a selected degree of flexibility; extension segments (or arms) S₂ having a lower degree of flexibility than central region S₁; and end segments (or feet) S₃ having a higher degree of flexibility than central region S₁. In essence, with this 5-zone construction, the central region S₁ provides a “saddle” for engaging the mitral valve annulus; the arms S₂ provide rigid structure for transferring the load to the outboard wall of the coronary sinus, a distance (along the axis of the vein) away from central region S₁; and the feet S₃ provide a “soft landing” for the load onto the outer wall of the coronary sinus. This 5-zone bar has been found to be a particularly advantageous construction inasmuch as (1) the 5-zone bar tends to center itself in the coronary sinus in position about the posterior leaflet of the mitral valve, in a sort of “macroelastic energy well”, whereby to minimize undesirable longitudinal bar migration; (2) the 5-zone bar tends to improve leaflet coaptation by reducing the distended mitral valve's anterior-to-posterior dimension without increasing the valve's commissure-to-commissure dimension, whereby to minimize the creation of undesirable “side jets”; and (3) the 5-zone bar has also been found to accommodate patient-to-patient anatomical variations extremely well.

In practice, each of treatment sections 305, 405 is also preferably formed with a tapered distal end 310, 410 (FIG. 24) terminating in an atraumatic ball tip 320, 420, such that treatment sections 305, 405 can be easily advanced from a location outside the patient's body into a working lumen 230 of delivery catheter 200 when shaft 205 is disposed in the coronary sinus of the patient. As a consequence of the foregoing tapered tip construction, each of treatment sections 305, 405 effectively has an additional distal end segment S₄ having a degree of flexibility even higher than the flexibility of the aforementioned end segments S₃.

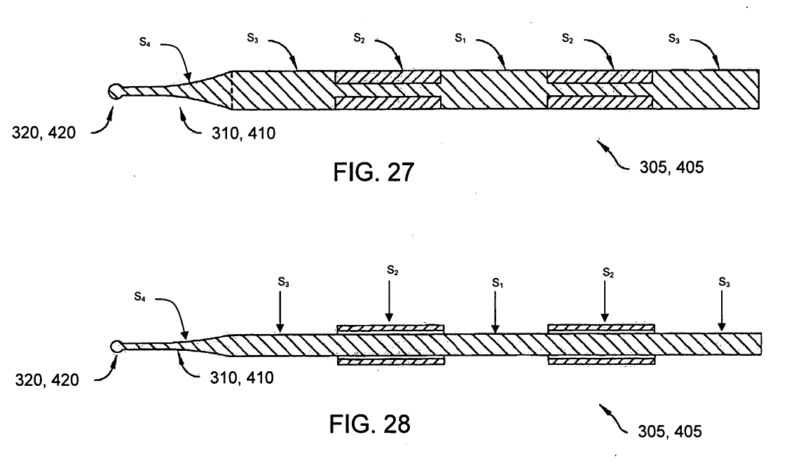

If desired, one or more of treatment sections 305, 405 may be formed out of a single piece of material (e.g., Nitinol), with the regions of differing flexibility S₁, S₂, S₃ and S₄ being provided by different rod diameters (see, for example, the construction shown in FIG. 25); and/or treatment sections 305, 405 may combine two or more different materials (e.g., stainless steel and Nitinol, etc.) in a composite construction (see, for example, the construction shown in FIG. 26, where the straightening rod comprises alternating sections of Nitinol and stainless steel, or the constructions shown in FIGS. 27 and 28, where the straightening rod comprises concentric arrangements of Nitinol and stainless steel) so as to provide treatment sections 305, 405 with the desired flexibility zones, etc.

As noted above, diagnostic rods 300 and treatment rods 400 have their treatment sections 305, 405 formed at the distal end of their shafts 310, 410. As also noted above, shafts 310, 410 correspond in length to one another, except that shaft 310 is longer than its counterpart shaft 410, and shaft 310 terminates in a handle 315 whereas shaft 410 terminates in a proximal tip 415. FIG. 22 shows details of the proximal end of shaft 310 and handle 315, and FIG. 23 shows details of the proximal end of shaft 410 and proximal tip 415. More particularly, as seen in FIGS. 3, 4 and 22, the diameter of handle 315 is significantly over-sized relative to the diameter of shaft 310, so as to permit a surgeon to easily grasp and manipulate diagnostic rod 300. Proximal tip 415, on the other hand, and looking now at FIGS. 3, 4 and 23, is actually reduced slightly in diameter relative to shaft 410. However, a peripheral enlargement 425 is formed just distal to proximal tip 415. Peripheral enlargement 425 has a diameter which is larger than the openings 284 in plate 283 of handle body 255, so as to limit distal movement of treatment rod 400 relative to delivery catheter 200, as will hereinafter be discussed.

### Pullback Device 500

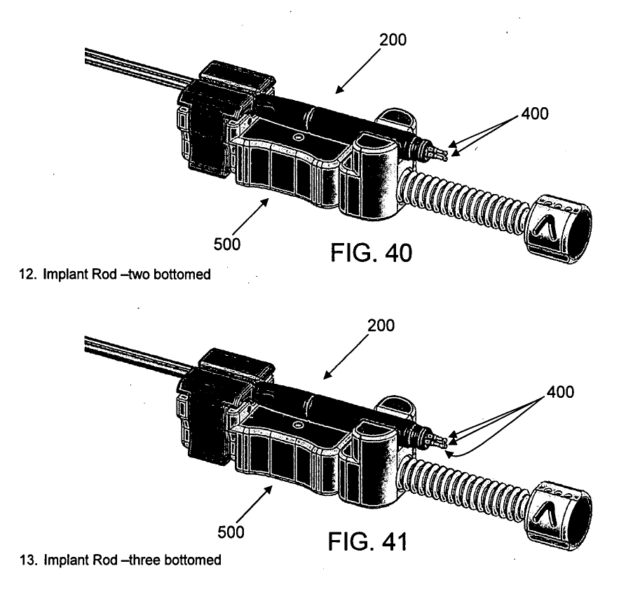

Annuloplasty system 100 also comprises a pullback device 500 for proper positioning of treatment rods 400 within delivery catheter 200. Looking next at FIGS. 3, 4 and 29-47, in one preferred form of the invention, pullback device 500 generally comprises a distal body 505, a threaded post 510 extending proximally from distal body 505 and rotatable with respect thereto, and a proximal body 515 movably mounted on threaded post 510.

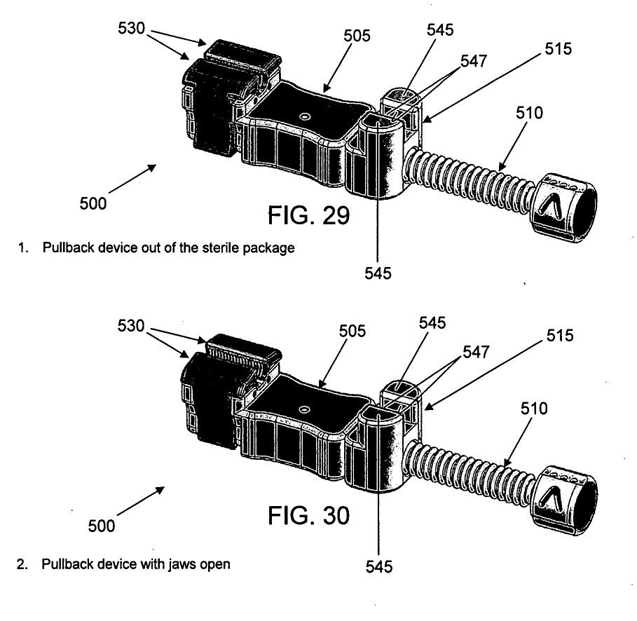

More particularly, distal body 510 has a bore 520 (FIG. 43) formed therein which rotatably receives threaded post 510 therein. A pin 525 locks threaded post 510 to distal body 505, while still permitting threaded post 510 to rotate with respect to distal body 505. A pair of jaws 530 (FIG. 30) are pivotally mounted to distal body 505, with jaws 530 being mounted to distal body 505 so as to be capable of assuming an open position (FIG. 30) and a closed position (FIG. 29).

Proximal body 515 comprises a threaded bore 535 (FIG. 43) which receives threaded post 510 therein. Thus, by turning a knob 540 formed on the proximal end of threaded post 510, proximal body 515 can ride on the threads of threaded post 510, whereby to cause proximal body 515 to move towards or away from distal body 505. Proximal body 515 also comprises a pair of raised posts 545 (FIG. 30), having a pair of opposing faces 547, for releasably engaging handle body 255 of handle 225, as will hereinafter be discussed in further detail.

Pullback device 500 is intended to be used for proper positioning of treatment rods 400 within delivery catheter 200.

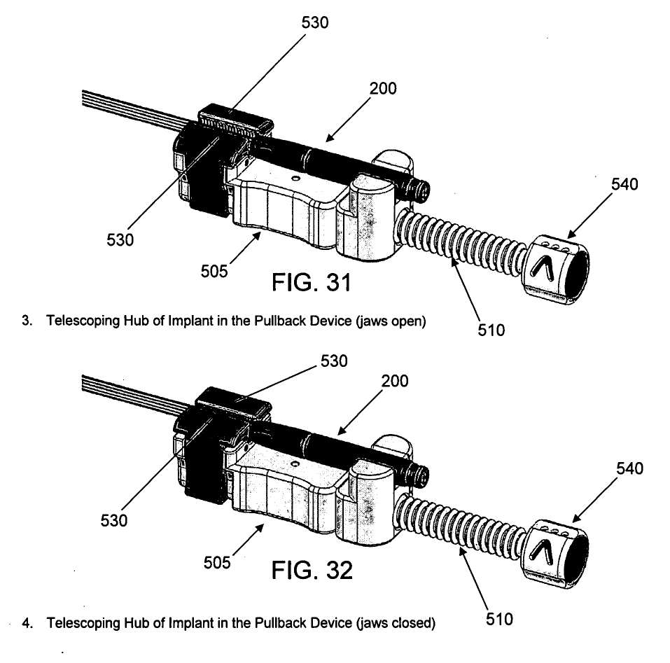

More particularly, in one preferred method of use, pullback device 500 is first set so that its proximal body 515 is positioned against its distal body 505 (FIG. 29) and jaws 530 are opened (FIG. 30). Then delivery catheter 200 (with its distal cap 260 in its loosened condition, and with its proximal cap 265 removed) is placed on pullback device 500 so that shaft 205 lies between opened jaws 530 and so that flats 282 of handle body 255 are engaged by opposing faces 547 of posts 545 (FIG. 31). Then jaws 530 are closed so as to grasp shaft 205 therebetween (FIG. 32). Thus, at this point, shaft 205 is secured to distal body 505 and handle body 255 is secured to proximal body 515, with distal cap 260 in its loosened condition so that fingers 272 are not grasping shaft 205.

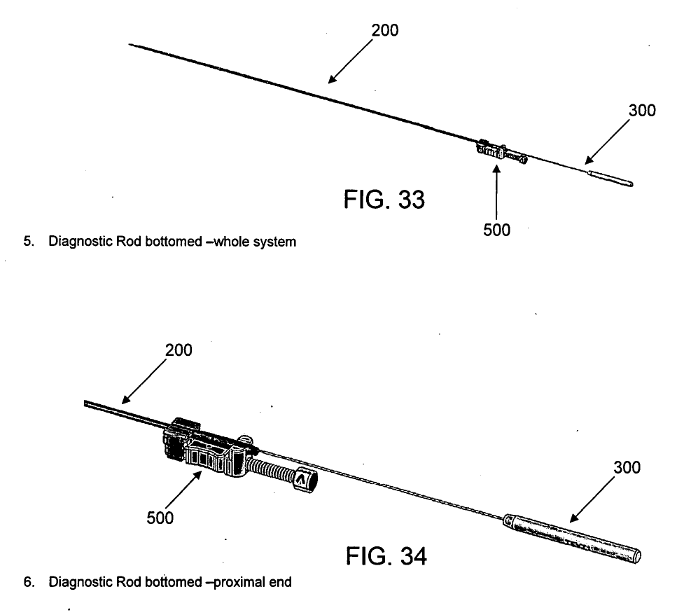

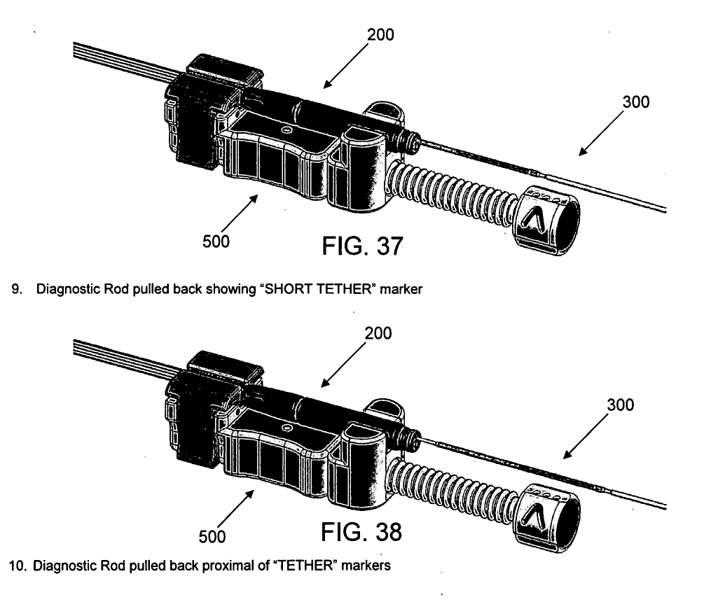

Next, a diagnostic rod 300 is inserted through an opening 284 formed in plate 283 of handle body 255 and then into a working lumen 230 of delivery catheter 200 (FIGS. 33-35). The diagnostic rod 300 is advanced distally within delivery catheter 200 and moved about, as will hereinafter be discussed in further detail, until treatment section 305 of diagnostic rod 300 is properly positioned within the patient's anatomy. The surgeon then reads indicia, formed on shaft 310 of diagnostic rod 300, vis-a-vis alignment with the proximal end of body 255 of handle 225 (FIGS. 36 and 37), so as to determine the appropriate length of the treatment rod 400 which is to thereafter be used for the patient. Then diagnostic rod 300 is withdrawn from delivery catheter 200 (FIG. 38).

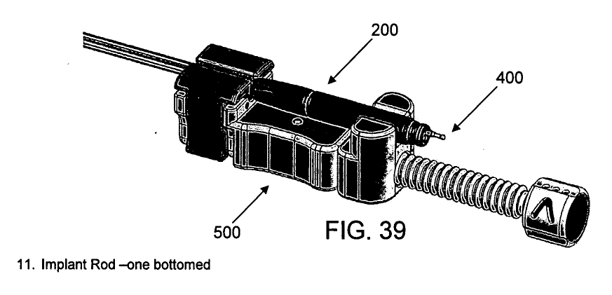

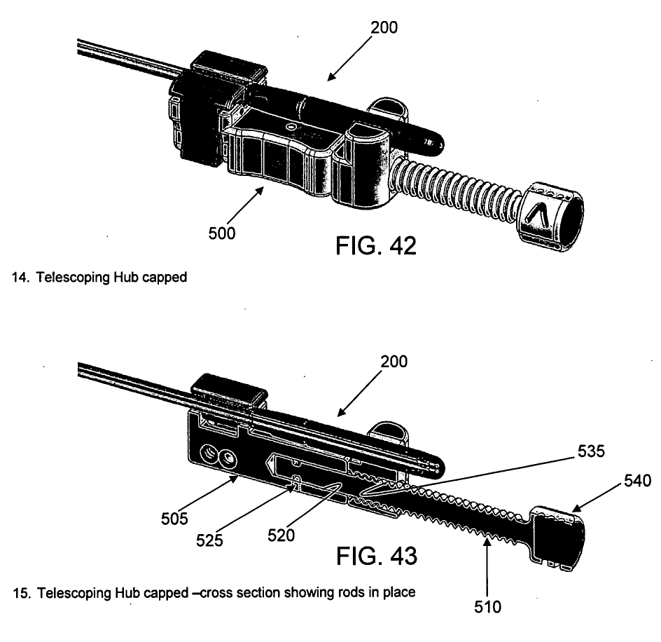

Next, one or more appropriately-sized treatment rods 400 are inserted into working lumens 230 of delivery catheter 200 (FIGS. 39-41). Preferably three treatment rods 400 are used, so as to fill the three working lumens 230 of delivery catheter 200. As treatment rods 400 are advanced along the coronary sinus, they impart the aforementioned straightening force to the coronary sinus. Treatment rods 400 are inserted until their peripheral enlargements 425 engage plate 283 of handle body 255. At this point, treatment sections 405 of treatment rods 400 will lie “over-distal” to the anatomy, i.e., treatment sections 405 will have moved past their optimal treatment locations within the anatomy, so that the distal ends of the treatment sections lie distal to the trigone AIV strongpoint 91 and the proximal ends of the treatment sections lie distal to the trigone CSO strongpoint 89, as will hereinafter be discussed in further detail. Then proximal cap 265 of handle 225 is secured to handle body 255 (FIGS. 42 and 43), whereby to capture peripheral enlargements 425 between plate 283 and the proximal cap, and thus capture treatment rods 400 to handle body 255. Again, distal cap 260 is in its loosened condition at this point in the procedure, so that fingers 272 are not grasping shaft 205.

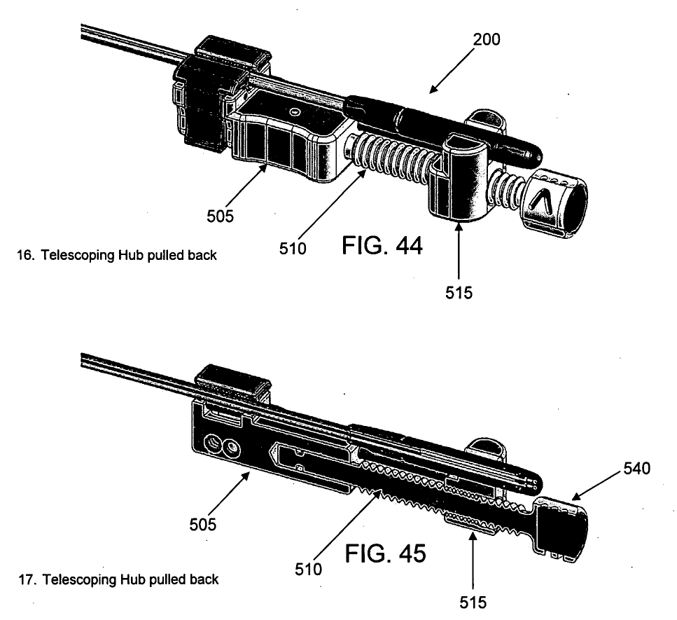

Then, while the surgeon observes the degree of patient regurgitation (e.g., using echocardiogram or other visualization methodology), knob 540 of pullback device 500 is turned so as to draw proximal body 515 away from distal body 505 (FIGS. 44 and 45). As this occurs, handle body 255 of handle 225 is withdrawn proximally along shaft 205, carrying with it treatment rods 400, whereby to draw the treatment sections 405 proximally relative to the anatomy. Knob 540 is turned until the “over-distal” treatment sections 405 are brought back, proximally, into their optimal treatment locations, i.e., so that the distal ends of the treatment sections are located adjacent to the trigone AIV strongpoint 91 and the proximal ends of the treatment sections lie adjacent to the trigone CSO strongpoint 89. This approach ensures that the posteriorly-directed forces generated by the distal and proximal ends of the treatment sections of the rods are carried by the trigone AIV strongpoint 91 and the trigone CSO strongpoint 89, such that appropriate anteriorly-directed forces can be generated by the intermediate portions of the rods, whereby to effect the desired valve remodeling. Thus, pullback device 500 can be used to properly position the treatment sections 405 relative to the anatomy, by drawing the “over-distal” treatment sections proximally back within the coronary sinus until they extend between the trigone AIV strongpoint 91 and the trigone CSO strongpoint 89, whereby to properly reconfigure the geometry of the mitral annulus, as will hereinafter be discussed in further detail.

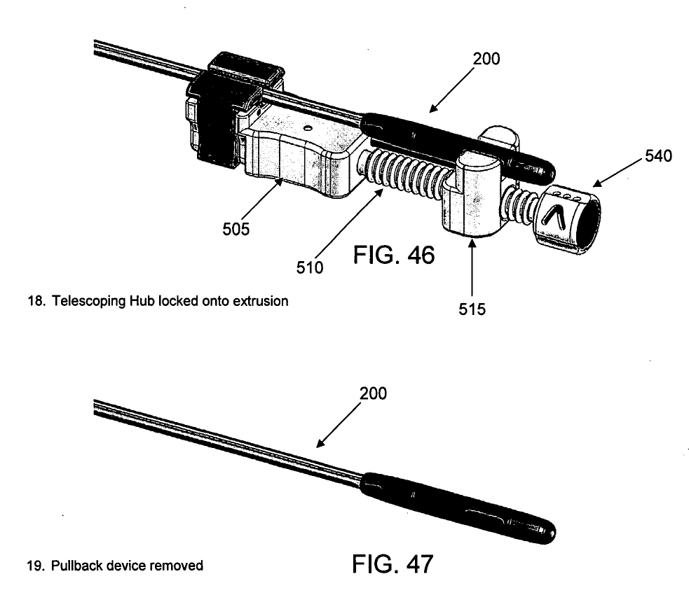

When handle body 255 has been moved an appropriate distance proximally so as to properly position treatment sections 405 of treatment rods 400 relative to the anatomy, distal cap 260 is screwed onto handle body 255, whereby to cause fingers 272 to grip shaft 205 and thereby secure handle 225 to shaft 205 (FIG. 46). Then pullback device 500 may be removed, leaving delivery catheter 200 properly deployed in the patient (FIG. 47).

Further details regarding the use of pullback device 500 in conjunction with the remainder of annuloplasty system 100 will hereinafter be discussed in further detail.

### Use

Annuloplasty system 100 is preferably used as follows.

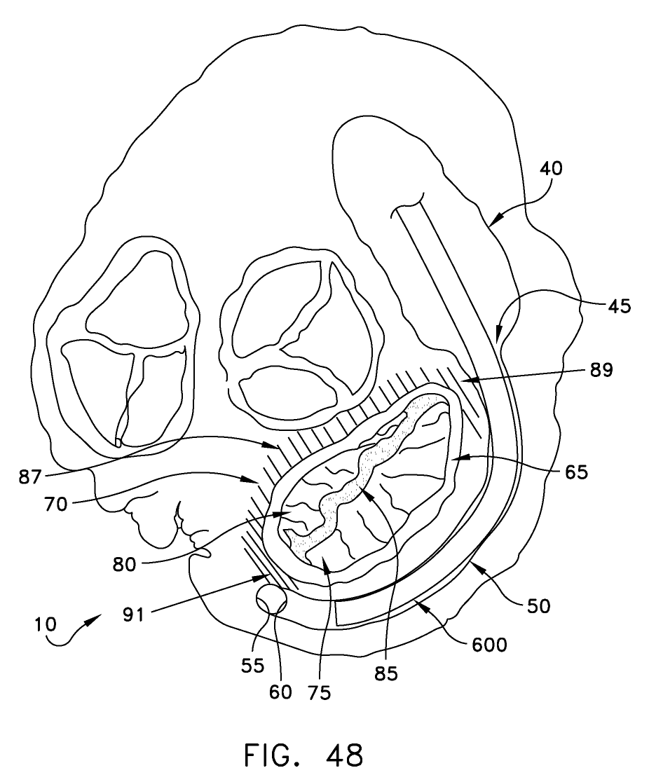

First, a standard introducer sheath of the sort well known in the art is introduced into the vascular system of the patient and advanced an appropriate distance into the coronary sinus. By way of example but not limitation, this may be accomplished by inserting the standard introducer sheath into the right or left subclavian vein of the patient, advancing it down the superior vena cava, through the right atrium of the heart, through the mouth of the coronary ostium and then down into the coronary sinus. In fact, the distal end of the standard introducer sheath is preferably advanced at least as far down the coronary sinus as traction mechanism 220 will extend into the coronary sinus, when delivery catheter 200 is thereafter deployed in the patient. This is important, since the standard introducer sheath provides a protective corridor for traction mechanism 220 during deployment of delivery catheter 200 within the patient, and ensures that traction mechanism 220 does not directly engage the vascular tissue of the patient until after delivery catheter 200 has been fully inserted into the patient and the standard introducer sheath is removed. See, for example, FIG. 48, which shows a standard introducer sheath 600 inserted into the coronary sinus of the patient.

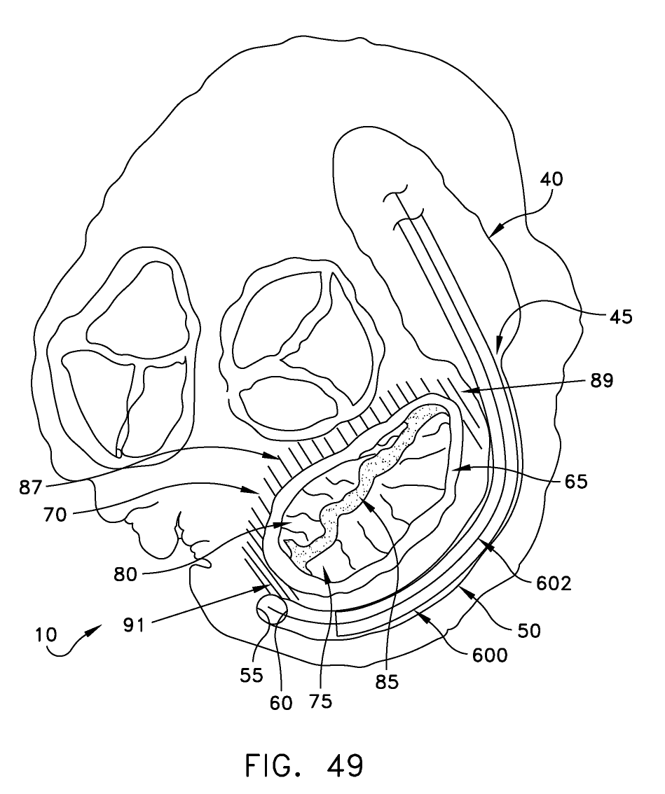

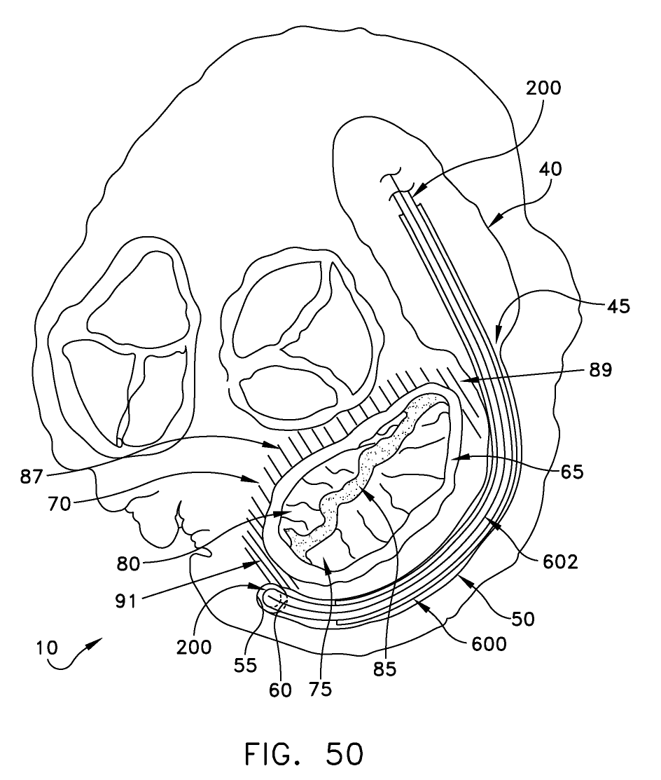

Then a guidewire 602 is advanced through standard introducer sheath 600 and into the coronary sinus of the patient (FIG. 49).

Next, delivery catheter 200 is loaded onto guidewire 602. Delivery catheter 200 is preferably loaded onto guidewire 602 by passing a working lumen 230 over the proximal end of the guidewire and then advancing delivery catheter 200 distally along the guidewire. Delivery catheter 200 is preferably loaded onto guidewire 602 after handle 225 of delivery catheter 200 has been mounted on pullback device 500, with distal cap 260 in its loosened condition and with proximal cap 265 removed, and with jaws 530 gripping shaft 205 and with flats 282 of body 255 engaging opposing faces 547 of posts 545.

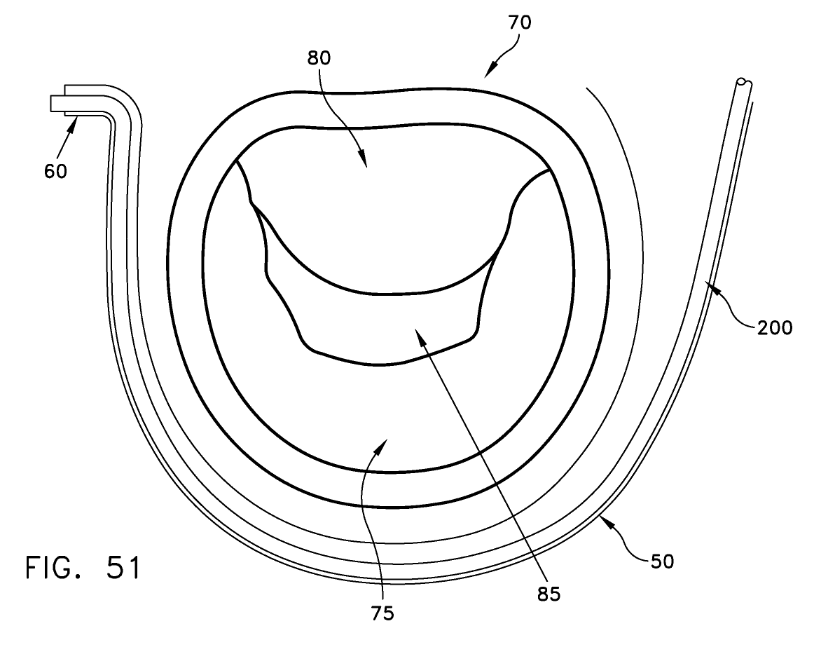

Delivery catheter is advanced distally down guidewire 602 until its distal end extends down into the AIV (FIGS. 50 and 51). As delivery catheter 200 is advanced into position, standard introducer sheath 600 acts as a shield between traction mechanism 220 and the adjacent tissue, thereby ensuring atraumatic deployment of delivery catheter 200 within the coronary sinus. Radiopaque markers 251 and/or 253 within shaft 205 may be used to help position delivery catheter 200 under fluoroscopy or the like. Additionally, if desired, a CT image of the patient's anatomy may be acquired prior to the procedure, and then during the procedure, the proper disposition of the delivery catheter may be determined by viewing the radio-opaque markers under fluoroscopy while simultaneously viewing the CT image, with the CT image being placed in co-registration with the fluoroscopic image. In one preferred form of the present invention, delivery catheter 200 is advanced until the proximal radio-opaque markers 251 (i.e., those just proximal to the proximal end of traction mechanism 220) are positioned at the coronary ostium 45, and so that traction mechanism 220 of delivery catheter 200 extends from the coronary ostium to the AIV, i.e., from the trigone CSO strongpoint 89 to the trigone AIV strongpoint 91. Again, as delivery catheter 200 is advanced, traction mechanism 220 remains shielded within standard introducer sheath 600 so as to atraumatically enter the patient.

Preferably, there are no diagnostic rods 300 or treatment rods 400 disposed in working lumens 230 of delivery catheter 200 while the delivery catheter is being advanced to the therapy site. As a result, inasmuch as shaft 205 is formed out of a relatively flexible material, shaft 205 will be able to readily flex as delivery catheter 200 is advanced into position, thereby further facilitating atraumatic device advancement. This is a significant advantage of the present invention, since it allows the delivery catheter to be deployed with a minimum of tissue trauma and with a reduced risk of device kinking.

Once delivery catheter 200 has been advanced into the vascular system of the patient so that its traction mechanism 220 extends adjacent to the posterior leaflet of the mitral valve and its distal tip extends down the AIV (FIGS. 50 and 51), guidewire 602 may be withdrawn.

Next, one or more diagnostic rods 300 are advanced down a working lumen 230 of delivery catheter 200. As diagnostic rods 300 are advanced along the coronary sinus, they impart the aforementioned straightening force to the coronary sinus. These diagnostic rods 300 are advanced distally within delivery catheter 200 until treatment sections 305 of diagnostic rods 300 are located well distal (i.e., “over-distal”) of their maximum treatment position, i.e., so that the distal ends of treatment sections 305 have advanced well beyond the trigone AIV strongpoint 91 and so that the proximal ends of treatment sections 305 have advanced well beyond the trigone CSO strongpoint 89.

It is desirable to initially position diagnostic rods 300 into this “over-distal” position, particularly when more than one diagnostic rod 300 is being used, since the “over-distal” position is anatomically stable and there is little risk of device migration.

One of the diagnostic rods is then pulled slowly proximally so as to draw the proximal end of its treatment section up “onto” the trigone CSO strongpoint 89, whereupon the distal end of its treatment section rests adjacent to AIV strongpoint 91. In this position the treatment section of the diagnostic rod has its maximum effect on the mitral valve, since the distal end of the treatment section applies its posteriorly-directed force on the coronary sinus in the area of the trigone AIV strongpoint 91 and the proximal end of the treatment section applies its posteriorly-directed force on the coronary sinus in the area of the trigone CSO strongpoint 89, whereby to permit the intermediate portion of the treatment rod to generate a sufficient anteriorly-directed force on the annulus of the mitral valve in order to effect the desired valve remodeling.

The ability of the system to initially place the diagnostic rods 300 (and, later, the treatment rods 400) in a very stable, “over-distal of best treatment position”, and thereafter carefully draw the diagnostic rods (and, later, the treatment rods) back (i.e., proximally) into optimal position is an important advantage of the system, since it allows the treatment sections to be reliably maneuvered proximally until they are safely placed in an anatomically-stable and therapeutic position, without subsequent undesired device migration.

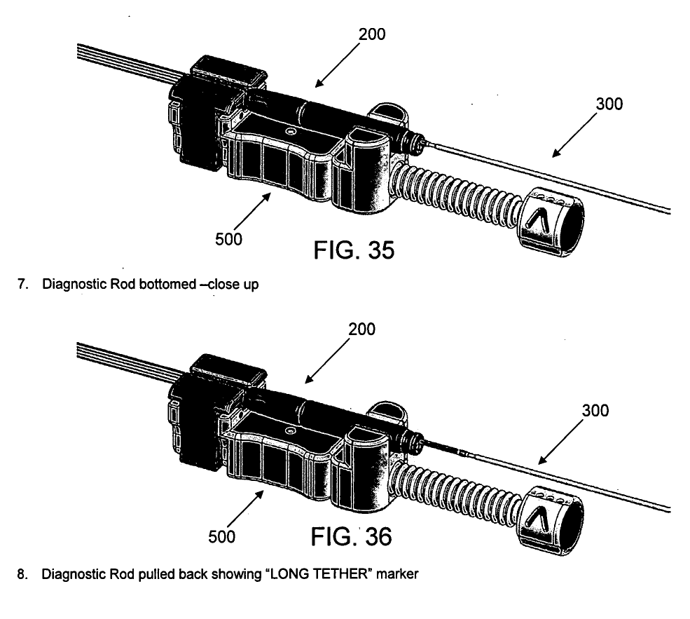

Once the diagnostic rod has been pulled proximally so that it is seated in its correct anatomical position, the surgeon then identifies the indicia aligned with the proximal end of handle body 255 of handle 225 (FIG. 35), and uses this information to determine the appropriate treatment rod 400 to be used for the patient. At this point standard introducer sheath 600 is partially split and partially withdrawn, so as to expose the wall of the coronary sinus to traction mechanism 220.

Then the one or more diagnostic rods 300 which are disposed within delivery catheter 200 are “swapped out” for one or more appropriately-sized treatment rods 400. Again, as treatment rods 400 are advanced along the coronary sinus, they impart the aforementioned straightening force to the coronary sinus. Treatment rods 400 are inserted into delivery catheter 200 until the peripheral enlargements 425 of treatment rods 400 engage plate 283 of handle body 255. At this point the treatment sections 405 will lie “over-distal”, i.e., treatment sections 405 will have moved past their optimal treatment locations, so that the distal ends of the treatment sections lie distal to the trigone AIV strongpoint 91 and the proximal ends of the treatment section lie distal to the trigone CSO strongpoint 89.

As each treatment rod 400 is inserted into a working lumen 230 of delivery catheter 200, shaft 205 becomes progressively stiffer and hence straighter, incrementally remodeling the geometry of the distended mitral valve so as to urge its posterior leaflet anteriorly, whereby to reduce mitral regurgitation (FIG. 50). Of course, at this point in the procedure, treatment rods 400 will not provide their full therapeutic effect, since they will be disposed “overly-distal” of their optimal treatment locations; nonetheless, they will still be providing substantial valve remodeling at this point in the procedure.

As each successive treatment rod 400 is inserted into a working lumen 230 of shaft 205, the degree of mitral valve regurgitation is observed, with the process continuing until the degree of regurgitation is minimized. As each successive treatment rod 400 is inserted into delivery catheter 200 and the tissue is incrementally loaded, traction mechanism 220 further engages the surrounding tissue, stabilizing the distal end of delivery catheter 200 in position relative to the tissue. Then proximal cap 265 is secured to the handle body 255. This captures treatment rods 400 relative to handle body 255, by capturing peripheral enlargements 425 of treatment rods 400 between plate 283 of handle body 255 and proximal cap 265.

Next, as the surgeon observes the degree of patient regurgitation (e.g., using echocardiogram or other visualization methodology), knob 540 of pullback device 500 is rotated so as to draw proximal body 515 away from distal body 505. As this occurs, handle body 255 of handle 225 is withdrawn proximally along shaft 205, simultaneously carrying with it the one or more treatment rods 400. However, such proximal movement of treatment rods 400 is independent of shaft 205 (and hence independent of the anatomy of the patient) since distal cap of handle 225 is in its unscrewed position, thereby allowing handle body 255 (and hence treatment rods 400) to move proximally relative to shaft 205. Thus, turning of knob 540 moves the position of treatment rods 400 proximally relative to the anatomy.

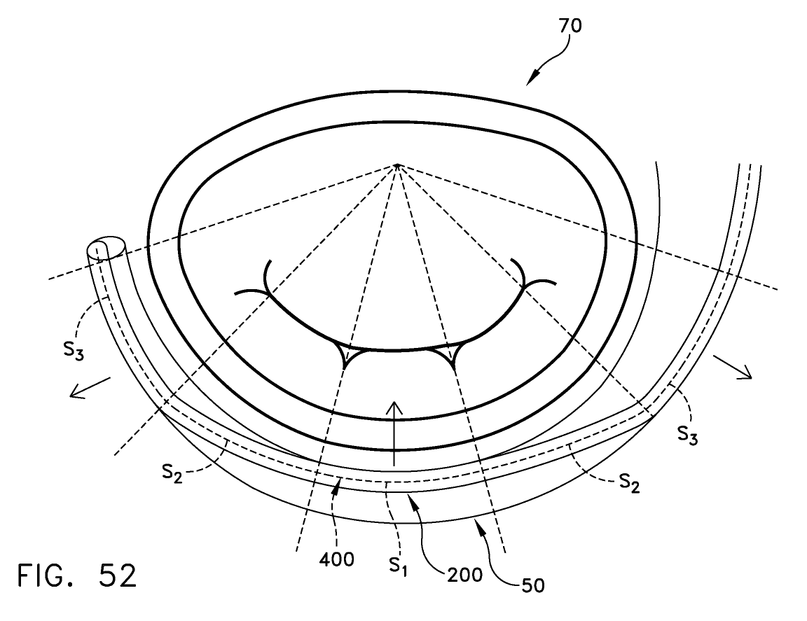

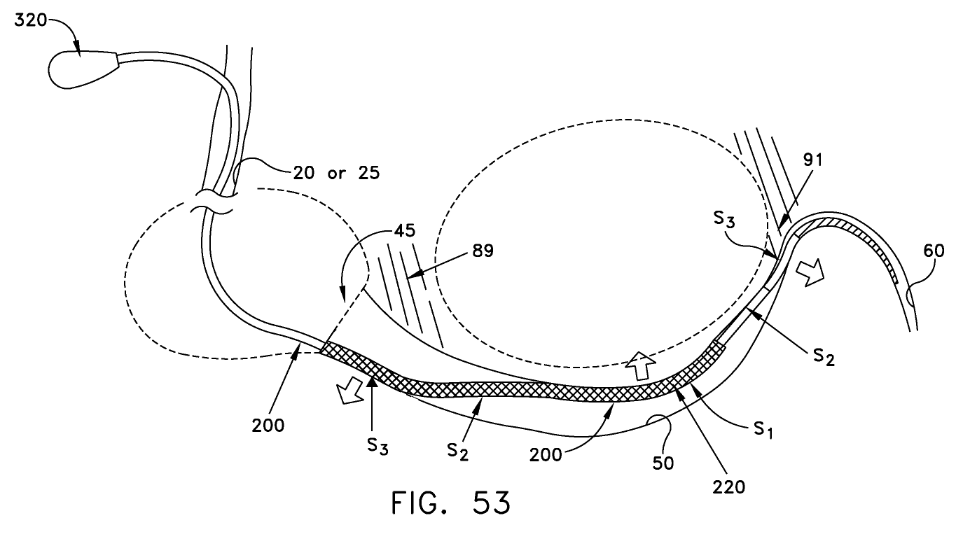

Preferably, knob 540 is moved a sufficient distance proximally to draw the “over-distal” treatment sections 405 back into their optimal treatment locations, i.e., so that the proximal ends of treatment sections 405 are located at trigone SCO strongpoint 89 and the distal ends of treatment sections 405 are located at trigone AIV strongpoint 91. This rod position permits treatment rods 400 to apply the maximum reconfiguration therapy to the anatomy, since the posteriorly-directed forces applied by the ends of treatment sections 405 are carried by the trigone CSO strongpoint 89 and the trigone AIV strongpoint 91, thereby permitting generation of the anteriorly-directed force needed for valve remodeling. See FIGS. 52 and 53. Thus, in accordance with the present invention, the treatment rods are first placed “over-distal” and then pulled back proximally so as to “lift” the proximal ends of the treatment rods up onto the tough fibrous tissue of the coronary ostium.

When handle body 255 has been moved the appropriate distance so as to properly position the treatment sections 405 of treatment rods 400 relative to the anatomy, distal cap 260 is screwed onto handle body 255, whereby to secure handle body 255 to shaft 205.

Thereafter, delivery catheter 200 is dismounted from pullback device 500, pullback device 500 is removed from the surgical site, handle 225 of delivery catheter 200 is positioned within a pocket formed in the torso of the patient (e.g., in the manner of a subcutaneous port), and then the pocket is closed, leaving annuloplasty system 100 deployed in the patient, with the intermediate portions of treatment sections 405 applying an anteriorly-directed force against the posterior side of the mitral valve annulus, and with the proximal and distal portions of treatment section 405 applying posteriorly-directed forces against the outer side wall of the coronary sinus.

Significantly, and looking now at FIG. 53, these posteriorly-directed forces are applied in the region of the trigone CSO strongpoint 89 and the trigone AIV strongpoint 91, so as to permit the generation of an appropriate anteriorly-directed force, whereby to reconfigure the annulus of the mitral valve and thereby reduce mitral regurgitation. In this respect it will be appreciated that the posteriorly-directed forces are not applied directly against the trigone CSO strongpoint 89 and the trigone AIV strongpoint 91, rather, the posteriorly-directed forces are actually applied directly away from the trigone CSO strongpoint 89 and the trigone AIV strongpoint 91. However, since the coronary sinus is strongly connected to the trigones at these two locations, the application of posteriorly-directed forces to the posterior side wall of the coronary sinus is substantially fully supported by the trigones. This in turn permits the generation of the necessary anteriorly-directed force which effects the desired valve remodeling.

Thus it will be seen that treatment rods 400 are sized and shaped so that they will induce a straightening of the coronary sinus when they are deployed in the coronary sinus. More particularly, each treatment section 405 of each treatment rod 400 is formed so as to be: (i) somewhat more rigid than the anatomical tissue surrounding the posterior leaflet of the mitral valve; and (ii) has a shape somewhat straighter than the natural curvature the patient's coronary sinus in the vicinity of the posterior leaflet of the mitral valve; and (iii) has an adequate length relative to the radius of curvature of the coronary sinus; such that when treatment rods 400 are disposed in the coronary sinus of the patient, they will impart a straightening force to the coronary sinus, so as to apply an anteriorly-directed force to the posterior leaflet of the mitral valve, whereby to reduce mitral regurgitation.

Significantly, shaft 205 of delivery catheter 200 may be constructed so that it, by itself, applies only a nominal straightening force to the wall of the coronary sinus. This arrangement can be highly advantageous, since it means that a shaft 205, lacking diagnostic rods 300 and/or treatment rods 400, can be easily and atraumatically advanced to the therapy site.

And, significantly, each treatment rod 400 need apply only a fraction of the total straightening force which is to be applied to the wall of the coronary sinus, since the cumulative effect of multiple treatment rods 400 may be harnessed. This is also highly advantageous, since it means that each individual treatment rod 400 may be easily and atraumatically advanced to the therapy site.

Also, significantly, by applying the straightening force to the mitral annulus through the use of one or more independently deployed treatment rods, different degrees of straightening force may be applied by using more or less treatment rods, and/or by using more or less rigid treatment rods, etc. In this respect it is noted that diagnostic rods 300 and treatment rods 400 are preferably provided in the form of a kit comprising a variety of different diagnostic rods 300 and treatment rods 400, each providing a different degree of straightening force, whereby to facilitate delivery of the optimal amount of tissue reconfiguration force.

If desired, a previously-emplaced treatment rod 400 may be removed, and/or replaced by a different treatment rod 400, so as to improve tissue reconfiguration and minimize mitral regurgitation. In essence, with treatment rods 400 being inserted into delivery catheter 200 while the delivery catheter is disposed in the coronary sinus, the final configuration of the annuloplasty system is essentially assembled in situ. This approach provides a number of significant advantages. Among other things, the serial insertion of treatment rods 400 into delivery catheter 200 allows the therapeutic treatment to be applied in a “stepwise fashion”, thereby allowing “fine tuning” of the tissue reconfiguration so as to enable optimal treatment.

Significantly, by forming each treatment rod 400 out of a resilient material, each treatment rod 400 need only apply a fraction of the force needed to effect substantially complete leaflet coaptation, inasmuch as treatment rod 400 can dynamically effect leaflet coaptation over time as the tissue progressively remodels. In this respect it should be noted that tissue tends to respond dynamically, so that a flexible treatment section 405 can be used to progressively drive the tissue closer and closer to a final position, whereby to effect tissue remodeling over a period of time, with the tissue being subjected to less trauma than if the desired tissue remodeling had been induced entirely at one time.

If desired, treatment rods 400 may also be pre-loaded into one or more working lumens 230 of shaft 205 prior to advancing delivery catheter 200 into the coronary sinus. However, as noted above, it is generally more desirable to load treatment rod 400 into working lumens 230 after delivery catheter 200 has been advanced into the coronary sinus, so that the delivery catheter will remain as flexible as possible during insertion into the coronary sinus of the patient.

If desired, treatment rods 400 may be formed out of a material able to accommodate the high strain imposed on treatment rods 400, e.g., a superelastic metal such as Nitinol.

In many situations it may be important to flush delivery catheter 200 with a fluid. This may be done to eliminate air emboli, or to provide a contrast medium, or for some other purpose. In this case, and looking now at FIGS. 5, 6 and 9, fluid may be introduced into one of the alignment stylets 290 so that the fluid will flow down the working lumen 230 associated with the alignment stylet, through window W into the adjacent working lumen 230, and then back out the alignment stylet associated with that second working lumen.

### Corridor System

As seen in FIG. 53, annuloplasty system 100 is constructed so that its delivery catheter 200 effectively creates an access “corridor” extending down to site of the therapy so as to permit atraumatic delivery of treatment rods 400. Significantly, delivery catheter 200 also provides a means for re-accessing the therapy site after the conclusion of the annuloplasty procedure. Thus, if it should subsequently be desired to adjust the degree of tissue reconfiguration, the same can be easily accomplished, e.g., by opening the tissue pocket so as to access handle 225 of delivery catheter 200, removing proximal cap 265, removing one or more of the previously-deployed treatment rods 400, installing one or more replacement treatment rods 400, and replacing proximal cap 265.

Alternatively, by providing an easy access corridor to the treatment site, the entire annuloplasty 100 can be subsequently removed from the patient if the same should be desired, i.e., by opening the tissue pocket so as to access the distal end of delivery catheter 200, removing proximal cap 265, removing treatment rods 400, and then removing delivery catheter 200.

### Modifications

It will be understood that many additional changes in the details, materials, steps and arrangements of parts, which have been herein described and illustrated in order to explain the nature of the invention, may be made by those skilled in the art within the principles and scope of the invention as expressed in the appended claims.

What is claimed is:

1. Treatment apparatus for reducing mitral regurgitation, the treatment apparatus comprising a treatment section which is sized and shaped for disposition within the coronary sinus of a patient, with the treatment section being formed so as to: (i) be somewhat more rigid than the anatomical tissue surrounding the posterior leaflet of the mitral valve; and (ii) have a shape somewhat straighter than the natural curvature of the coronary sinus in the vicinity of the posterior leaflet of the mitral valve; and (iii) have a length sufficient to span from the trigone CSO strongpoint to the trigone AIV strongpoint; such that when the treatment apparatus is placed in the coronary sinus of the patient, the treatment section applies posteriorly-directed forces to the outer wall of the coronary sinus in the vicinity of the trigone CSO strongpoint and the trigone AIV strongpoint, and the treatment section applies an anteriorly-directed force to the posterior annulus of the mitral valve, whereby to reconfigured the mitral valve and thereby reduce mitral regurgitation.

2. Apparatus according to claim 1 wherein the treatment apparatus further comprises a delivery catheter for providing a delivery corridor through which the treatment section is advanced when the treatment section is being deployed in the coronary sinus.

3. Apparatus according to claim 2 wherein the delivery catheter comprises a traction mechanism on an outer surface thereof for engaging the side wall of the coronary sinus between the trigone CSO strongpoint and the trigone AIV strongpoint.

4. Apparatus according to claim 3 wherein the traction mechanism comprises a helical winding projecting outboard from the outer surface of the delivery catheter.

5. Apparatus according to claim 2 wherein the treatment apparatus further comprises a standard introducer sheath, and further wherein the standard introducer sheath provides a delivery corridor through which the delivery catheter is advanced when the delivery catheter is deployed in the coronary sinus.

6. Apparatus according to claim 5 wherein the standard introducer sheath is at least partially split and partially withdrawn after the delivery catheter has been deployed in the coronary sinus and after the treatment section has been deployed in the coronary sinus.

7. Apparatus according to claim 6 wherein the standard introducer sheath is split along its longitudinal axis.

8. Apparatus according to claim 2 wherein the delivery catheter comprises at least one radio-opaque marker for identifying the position of the delivery catheter within the coronary sinus.

9. Apparatus according to claim 8 wherein the delivery catheter is configured so that the distal end of the delivery catheter can extend into the AIV when the at least one radio-opaque marker is substantially aligned with the coronary ostium.

10. Apparatus according to claim 9 further comprising a CT image of the patient's anatomy, such that the proper disposition of the delivery catheter may be determined by viewing the at least one radio-opaque marker under fluoroscopy and against a co-registered CT image.

11. Apparatus according to claim 2 wherein the apparatus is configured so that the treatment section can be moved to its proper location within the coronary sinus after the delivery catheter has been positioned in the coronary sinus and without moving the delivery catheter within the coronary sinus.

12. Apparatus according to claim 11 wherein the apparatus is configured so that the treatment section can be positioned in the coronary sinus by first advancing the treatment section to an “over-distal” position and then retracting the treatment section proximally into the desired position.

13. Apparatus according to claim 12 wherein (i) when the treatment section is in the “over-distal position”, the distal end of the treatment section resides distal to the trigone AIV strongpoint and the proximal end of the treatment section resides distal to the trigone CSO strongpoint, and (ii) when the treatment section is in the desired position, the distal end of the treatment section resides approximately adjacent to the trigone AIV strongpoint and the proximal end of the treatment section resides approximately adjacent to the trigone CSO strongpoint.

14. Apparatus according to claim 1 wherein the treatment section comprises a resilient material.

15. Apparatus according to claim 14 wherein the treatment section comprises a superelastic shape memory alloy.

16. Apparatus according to claim 15 wherein the treatment section comprises Nitinol.

17. Apparatus according to claim 1 wherein a plurality of treatment sections are disposed in the coronary sinus.

18. Apparatus according to claim 17 wherein one of the plurality of treatment sections is disposed in the coronary sinus before a second of the treatment sections is disposed within the coronary sinus.

19. Apparatus according to claim 17 wherein the plurality of treatment sections are disposed parallel to one another in the coronary sinus.

20. A method for reducing mitral regurgitation, the method comprising the steps of:
providing treatment apparatus comprising a treatment section which is sized and shaped for disposition within the coronary sinus of a patient, with the treatment section being formed so as to: (i) be somewhat more rigid than the anatomical tissue surrounding the posterior leaflet of the mitral valve; and (ii) have a shape somewhat straighter than the natural curvature of the coronary sinus in the vicinity of the posterior leaflet of the mitral valve; and (iii) have a length sufficient to span from the trigone CSO strongpoint to the trigone AIV strongpoint; and
deploying the treatment apparatus in the coronary sinus of the patient, such that the treatment section applies posteriorly-directed forces to the outer wall of the coronary sinus in the vicinity of the trigone CSO strongpoint and the trigone AIV strongpoint, and the treatment section applies an anteriorly-directed force to the posterior annulus of the mitral valve, whereby to reconfigure the mitral valve and thereby reduce mitral regurgitation.

21. A method according to claim 20 wherein the treatment apparatus further comprises a delivery catheter for providing a delivery corridor through which the treatment section is advanced when the treatment section is being deployed in the coronary sinus.

22. A method according to claim 21 wherein the delivery catheter comprises a traction mechanism on an outer surface thereof for engaging the side wall of the coronary sinus between the trigone CSO strongpoint and the trigone AIV strongpoint.

23. A method according to claim 22 wherein the traction mechanism comprises a helical winding projecting outboard from the outer surface of the delivery catheter.

24. A method according to claim 21 wherein the treatment apparatus further comprises a standard introducer sheath, and further wherein the standard introducer sheath provides a delivery corridor through which the delivery catheter is advanced when the delivery catheter is deployed in the coronary sinus.

25. A method according to claim 24 wherein the standard introducer sheath is at least partially split and partially withdrawn after the delivery catheter has been deployed in the coronary sinus and after the treatment section has been deployed in the coronary sinus.

26. A method according to claim 25 wherein the standard introducer sheath is split along its longitudinal axis.

27. A method according to claim 20 wherein the delivery catheter comprises at least one radio-opaque marker for identifying the position of the delivery catheter within the coronary sinus.

28. A method according to claim 27 wherein the delivery catheter is configured so that the distal end of the delivery catheter can extend into the AIV when the at least one radio-opaque marker is substantially aligned with the coronary ostium.

29. A method according to claim 28 further comprising a CT image of the patient's anatomy, such that proper disposition of the delivery catheter may be determined by viewing the at least one radio-opaque marker under fluoroscopy and against a co-registered CT image.

30. A method according to claim 20 wherein the treatment section is moved to its proper location within the coronary sinus after the delivery catheter has been positioned in the coronary sinus and without moving the delivery catheter within the coronary sinus.

31. A method according to claim 30 wherein the treatment section is positioned in the coronary sinus by first moving it to an “over-distal” position and then retracting the treatment section proximally into the desired position.

32. A method according to claim 31 wherein (i) when the treatment section is in the “over-distal position”, the distal end of the treatment section resides distal to the trigone AIV strongpoint and the proximal end of the treatment section resides distal to the trigone CSO strongpoint, and (ii) when the treatment section is in the desired position, the distal end of the treatment section resides approximately adjacent to the trigone AIV strongpoint and the proximal end of the treatment section resides approximately adjacent to the trigone CSO strongpoint.

33. A method according to claim 20 wherein the treatment section comprises a resilient material.

34. A method according to claim 33 wherein the treatment section comprises a superelastic shape memory alloy.

35. A method according to claim 34 wherein the treatment section comprises Nitinol.

36. A method according to claim 20 wherein a plurality of treatment sections are disposed in the coronary sinus.

37. A method according to claim 36 wherein one of the plurality of treatment sections is disposed in the coronary sinus before a second of the treatment sections is disposed within the coronary sinus.

38. A method according to claim 36 wherein the plurality of treatment sections are disposed parallel to one another in the coronary sinus.

39. An assembly for reducing mitral regurgitation, the assembly comprising:
a delivery catheter comprising:
a shaft formed out of a material sufficiently flexible to assume a first configuration generally conforming to a coronary sinus upon insertion of the shaft into the coronary sinus, and to assume a straighter second configuration when biased toward the straighter configuration, the shaft having at least one lumen extending lengthwise therethrough;
a handle slidably mounted on the shaft, the handle comprising an end plate having at least one opening therein for alignment with the at least one lumen of the shaft; and
a collet mechanism attached to the handle for fixing the position of the handle relative to the shaft;
at least one treatment rod comprising a treatment section, a rod shaft and a peripheral enlargement disposed at the proximal end of the rod shaft;
the treatment section and the rod shaft being sized to fit within a lumen in the shaft and an opening in the end plate of the handle, and the peripheral enlargement being sized larger than the opening in the end plate of the handle so as to limit distal movement of the treatment rod relative to the delivery catheter;
the treatment section being formed so as to (i) be more rigid than the anatomical tissue surrounding the posterior leaflet of the mitral valve;
and (ii) have a shape straighter than the shape of the coronary sinus in the vicinity of the posterior leaflet of the mitral valve; and (iii) have an adequate length relative to the radius of curvature of the coronary sinus, such that when the at least one treatment rod is advanced down the lumen while the shaft is positioned in the coronary sinus adjacent to the posterior leaflet of the mitral valve, the treatment section will impart a straightening force to the wall of the coronary sinus, whereby to move the posterior annulus anteriorly so as to improve leaflet coaptation and, as a result, reduce mitral regurgitation; and
a pullback device mountable to the delivery catheter for adjusting the position of the handle along the shaft before the collet mechanism is used to fix the position of the handle along the shaft.

40. An assembly according to claim 39 wherein the pullback device comprises a first mechanism for gripping the shaft and a second mechanism for gripping the handle, and further wherein the first mechanism is movable relative to the second mechanism.

41. An assembly according to claim 40 wherein the shaft has a traction mechanism secured thereto, wherein the traction mechanism projects laterally outboard of the shaft so as to mechanically engage tissue.

42. An assembly according to claim 41 wherein the traction mechanism comprises a helical coil wrapped about the exterior of the shaft.

43. An assembly according to claim 39 wherein the treatment section is provided with varying degrees of stiffness along the length thereof.

44. An assembly according to claim 39 wherein the treatment section comprises first and second end portions connected together by an intermediate portion, wherein the intermediate portion comprises first and second regions connected together by a central region, wherein the central region and the first and second end portions are substantially curved after the elongated body is inserted into the coronary sinus, and further wherein the first and second regions are substantially straight after the elongated body is inserted into the coronary sinus.

45. An assembly according to claim 44 wherein the first and second regions are stiffer than the central region, and further wherein the central region is stiffer than the first and second end portions.

46. An assembly according to claim 44 wherein the central region, the first and second end portions and the first and second regions have a length such that the elongated body applies an anteriorly-directed force to the walls of the coronary sinus substantially adjacent to the posterior leaflet of the valve, and applies posteriorly-directed forces to the walls of the coronary sinus substantially adjacent to the trigone AIV strongpoint and the trigone CSO strongpoint.

47. An assembly according to claim 39 wherein the treatment sections are formed at least in part out of a resilient material.

48. An assembly according to claim 39 wherein the treatment sections are formed at least in part out of a superelastic material.

49. An assembly according to claim 39 wherein the assembly further comprises a guidewire, and the delivery catheter further comprises an opening through which the guidewire is movable.

50. An assembly according to claim 49 wherein the opening comprises one of the lumens.

51. An assembly according to claim 39 wherein the treatment section is substantially straight in an unstressed condition.

52. An assembly according to claim 39 wherein the treatment section is substantially curved after insertion into the coronary sinus.
